(19) United States
(12) Patent Application Publication
(10) Pub. No.: US 2024/0207842 A1
(43) Pub. Date: Jun. 27, 2024

(54) FLOW CELLS

(71) Applicant: ILLUMINA, INC., San Diego, CA (US)

(72) Inventors: Gianluca Andrea Artioli, Cambridge (GB); Johan Sebastian Basuki, Singapore (SG); Boyan Boyanov, San Diego, CA (US); Gabriele Canzi, San Diego, CA (US); Sahngki Hong, San Diego, CA (US); Nam Nguyen, Cambridge (GB); Sebastien Georg Gabriel Ricoult, Cambridge (GB); Alexandra Szemjonov, Cambridge (GB); Xavier von Hatten, Cambridge (GB)

(21) Appl. No.: 18/542,511

(22) Filed: Dec. 15, 2023

Related U.S. Application Data
(60) Provisional application No. 63/601,655, filed on Nov. 21, 2023.
(60) Provisional application No. 63/586,716, filed on Sep. 29, 2023.
(60) Provisional application No. 63/387,874, filed on Dec. 16, 2022.

(51) Int. Cl. B01L 3/00

(52) U.S. Cl. CPC B01L 3/5027; B01L 2200/025; B01L 2300/12; B01L 2300/16; B01L 2300/1805

(57) ABSTRACT

An example of a flow cell includes a substrate; a plurality of reactive regions spatially separated from one another across the substrate; and a plurality of independently removable coatings respectively positioned over each of the plurality of reactive regions. Each of the plurality of reactive regions includes a polymeric hydrogel layer; and a reactive entity attached to the polymeric hydrogel layer. At least one of the independently removable coatings is a gas-dissolvable coating.

### CROSS-REFERENCE TO RELATED APPLICATIONS

This application claims the benefit of U.S. Provisional Patent Application 63/601,655, filed Nov. 21, 2023, and U.S. Provisional Patent Application Ser. No. 63/586,716, filed Sep. 29, 2023, and U.S. Provisional Patent Application Ser. No. 63/387,874, filed Dec. 16, 2022, the contents of each of which are incorporated herein by reference in its entirety.

### REFERENCE TO SEQUENCE LISTING

The Sequence Listing submitted herewith is hereby incorporated by reference in its entirety. The name of the file is ILI251B3_IP-2712-US_Sequence_Listing.xml, the size of the file is 17,866 bytes, and the date of creation of the file is Dec. 11, 2023.

### BACKGROUND

Some biological and/or chemical vessels, such as assay plates and flow cells, include designated reaction areas, where surface chemistry that enables a desired interaction or reaction is localized. When a reactive species is introduced into the vessel, the reactive species interacts or reacts with the surface chemistry to create a detectable signal (e.g., an electrical signal or an optical signal). Many vessels are configured with multiple reaction areas in fluid communication with a single flow channel. In these vessels, a single sample may be introduced into the flow channel and its associated reaction areas, or multiple samples may be pooled and introduced into the flow channel and its associated reaction areas.

### SUMMARY

The biological and/or chemical vessels disclosed herein include a plurality of reactive regions spatially separated from one another across a substrate. These reactive regions include respective reactive entities that may be the same or different.

Each reactive region may be coated with an independently removable protective coating, which may a gas-dissolvable coating or a heat-responsive coating. These independently removable coatings enable controlled access to the reactive regions. For example, one or more coating(s) may be removed via exposure to heat that is generated by a heating mechanism, while one or more other coating(s) remain(s) intact. As another example, one or more coating(s) may be removed via exposure to a reactive gas, while one or more other coating(s) remain(s) intact. The reactive region(s) exposed by coating removal become(s) active and thus is/are able to participate in the designated reaction. The reactive region(s) having its/their coating(s) intact remain passivated or protected, and thus remain(s) inactive.

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1A is a top view of a flow cell;

FIG. 1B is a semi-schematic, partially cross-sectional and partially

perspective view an example architecture of the flow cell of FIG. 1A;

FIG. 1C is a semi-schematic, partially cross-sectional and partially perspective view of another example architecture of the flow cell of FIG. 1A;

FIG. 2A depicts a chemical structure of one example of a gas-dissolvable protective coating and a reaction involving the gas-dissolvable protective coating;

FIG. 2B depicts a chemical structure of another example of the gas-dissolvable protective coating and a reaction involving the gas-dissolvable protective coating;

FIG. 3 depicts a chemical structure of an example of a heat-responsive protective coating and a reaction involving the heat-responsive protective coating;

FIG. 4 is a schematic illustration of a flow cell including a complementary metal-oxide semiconductor (CMOS) imaging device that is coupled to a substrate;

FIG. 5 schematically illustrates two example methods (A., B., C., D., E. or A., B., F., G., E.) utilizing gas-dissolvable or heat-responsive protective coatings, where:

A. illustrates the exposure of one gas-dissolvable or heat-responsive protective coating to removal conditions, B. illustrates the introduction of a first library template strand, C. illustrates the seeding of the first library template strand and the exposure of another gas-dissolvable or heat-responsive coating to removal conditions, D. illustrates the introduction of a second library template strand, and E. illustrates the amplified template strands;

A. illustrates the exposure of one gas-dissolvable or heat-responsive coating to removal conditions, B. illustrates the introduction of a first library template strand, F. illustrates the seeding and amplification of the first library template strand, and the exposure of another gas-dissolvable or heat-responsive protective coating to removal conditions, G. illustrates the introduction of a second library template strand, and E. illustrates the amplified template strands;

FIG. 6A is a schematic cross-sectional view of reactive areas defined on protrusions and coated with different removable coatings, some of which include multiple sub-layers; and

FIG. 6B is a schematic cross-sectional view of reactive areas defined on protrusions and coated with the same removable coating having different thicknesses.

### Definitions

It is to be understood that terms used herein will take on their ordinary meaning in the relevant art unless specified otherwise. Several terms used herein and their meanings are set forth below.

The singular forms “a”, “an”, and “the” include plural referents unless the context clearly dictates otherwise.

The terms comprising, including, containing and various forms of these terms are synonymous with each other and are meant to be equally broad.

The terms top, bottom, lower, upper, on, etc. are used herein to describe the flow cell and/or the various components of the flow cell. It is to be understood that these directional terms are not meant to imply a specific orientation, but are used to designate relative orientation between components. The use of directional terms should not be interpreted to limit the examples disclosed herein to any specific orientation(s).

The terms first, second, etc. also are not meant to imply a specific orientation or order, but rather are used to distinguish one component from another.

It is to be understood that the ranges provided herein include the stated range and any value or sub-range within the stated range, as if such values or sub-ranges were explicitly recited. For example, a range of about 400 nm to about 1 μm (1000 nm), should be interpreted to include not only the explicitly recited limits of about 400 nm to about 1 μm, but also to include individual values, such as about 708 nm, about 945.5 nm, etc., and sub-ranges, such as from about 425 nm to about 825 nm, from about 550 nm to about 940 nm, etc. Furthermore, when “about” and/or “substantially” are/is utilized to describe a value, they are meant to encompass minor variations (up to +/−10%) from the stated value.

An “acrylamide monomer” is a monomer with the structure

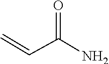

or a monomer including an acrylamide group. Examples of the monomer including an acrylamide group include azido acetamido pentyl acrylamide:

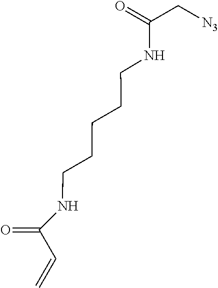 [SMILES: C=CC(=O)NCCCCCNC(=O)CN=[N+]=[N-]]

### and N-isopropylacrylamide:

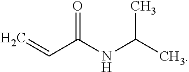

Other acrylamide monomers may be used.

The term “activation,” as used herein, refers to a process that generates reactive groups at the surface of a substrate. Activation may be accomplished using silanization or plasma ashing. While the figures do not depict a separate silanized layer or hydroxyl (—OH groups) from plasma ashing, it is to be understood that activation generates a silanized layer or —OH groups at the surface of the activated support or layer to covalently attach the functionalized layers to the underlying support or layer.

An aldehyde, as used herein, is an organic compound containing a functional group with the structure —CHO, which includes a carbonyl center (i.e., a carbon double-bonded to oxygen) with the carbon atom also bonded to hydrogen and an R group, such as an alkyl or other side chain. The general structure of an aldehyde is:

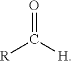

As used herein, “alkyl” refers to a straight or branched hydrocarbon chain that is fully saturated (i.e., contains no double or triple bonds). The alkyl group may have 1 to 20 carbon atoms. Example alkyl groups include methyl, ethyl, propyl, isopropyl, butyl, isobutyl, tertiary butyl, pentyl, hexyl, and the like. As an example, the designation “C1-4 alkyl” indicates that there are one to four carbon atoms in the alkyl chain, i.e., the alkyl chain is selected from the group consisting of methyl, ethyl, propyl, iso-propyl, n-butyl, isobutyl, sec-butyl, and t-butyl.

As used herein, “alkenyl” refers to a straight or branched hydrocarbon chain containing one or more double bonds. The alkenyl group may have 2 to 20 carbon atoms. Example alkenyl groups include ethenyl, propenyl, butenyl, pentenyl, hexenyl, and the like.

As used herein, “alkyne” or “alkynyl” refers to a straight or branched hydrocarbon chain containing one or more triple bonds. The alkynyl group may have 2 to 20 carbon atoms.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms. Examples of aryl groups include phenyl, naphthyl, azulenyl, and anthracenyl.

An “amine” or “amino” functional group refers to an —NRₐR_{b} group, where Rₐ and R_{b} are each independently selected from hydrogen

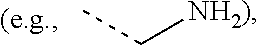

C1-6 (or C1-C6) alkyl, C2-6 alkenyl, C2-6 alkynyl, C3-7 carbocycle, C6-10 aryl, 5-10 membered heteroaryl, and 5-10 membered heterocyclyl, as defined herein.

As used herein, the term “attached” refers to the state of two things being joined, fastened, adhered, connected or bound to each other, either directly or indirectly. For example, a nucleic acid can be attached to a polymeric hydrogel layer by a covalent or non-covalent bond. A covalent bond is characterized by the sharing of pairs of electrons between atoms. A non-covalent bond is a physical bond that does not involve the sharing of pairs of electrons and can include, for example, hydrogen bonds, ionic bonds, van der Waals forces, hydrophilic interactions and hydrophobic interactions.

An “azide” or “azido” functional group refers to —N₃.

As used herein, a “bonding region” refers to an area of a patterned structure that is to be bonded to another material, which may be, as examples, a spacer layer, a lid, another patterned structure, etc., or combinations thereof (e.g., a spacer layer and a lid, or a spacer layer and another patterned structure). The bond that is formed at the bonding region may be a chemical bond, or a mechanical bond (e.g., using a fastener, etc.).

As used herein, “carbocycle” means a non-aromatic cyclic ring or ring system containing only carbon atoms in the ring system backbone. When the carbocycle is a ring system, two or more rings may be joined together in a fused, bridged or spiro-connected fashion. Carbocycles may have any degree of saturation, provided that at least one ring in a ring system is not aromatic. Thus, carbocycles include cycloalkyls, cycloalkenyls, and cycloalkynyls. The carbocycle group may have 3 to 20 carbon atoms. Examples of carbocycle rings include cyclopropyl, cyclobutyl, cyclopentyl, cyclohexyl, cyclohexenyl, 2,3-dihydro-indene, bicyclo[2.2.2]octanyl, adamantyl, and spiro[4.4]nonanyl.

As used herein, the term “carboxylic acid” or “carboxyl” refers to —COOH.

As used herein, “cycloalkylene” means a fully saturated carbocycle ring or ring system that is attached to the rest of the molecule via two points of attachment.

As used herein, “cycloalkenyl” or “cycloalkene” means a carbocycle ring or ring system having at least one double bond, wherein no ring in the ring system is aromatic. Examples include cyclohexenyl or cyclohexene and norbornenyl or norbornene. Also as used herein, “heterocycloalkenyl” or “heterocycloalkene” means a carbocycle ring or ring system with at least one heteroatom in ring backbone, having at least one double bond, wherein no ring in the ring system is aromatic.

As used herein, “cycloalkynyl” or “cycloalkyne” means a carbocycle ring or ring system having at least one triple bond, wherein no ring in the ring system is aromatic. An example is cyclooctyne. Another example is bicyclononyne. Also as used herein, “heterocycloalkynyl” or “heterocycloalkyne” means a carbocycle ring or ring system with at least one heteroatom in ring backbone, having at least one triple bond, wherein no ring in the ring system is aromatic.

The term “depositing,” as used herein, refers to any suitable application technique, which may be manual or automated, and, in some instances, results in modification of the surface properties. Generally, depositing may be performed using vapor deposition techniques, coating techniques, grafting techniques, or the like. Some specific examples include chemical vapor deposition (CVD), spray coating (e.g., ultrasonic spray coating), spin coating, dunk or dip coating, doctor blade coating, puddle dispensing, flow through coating, aerosol printing, screen printing, microcontact printing, inkjet printing, or the like.

As used herein, the term “depression” refers to a discrete concave feature, defined in a substrate, and having a surface opening that is at least partially surrounded by interstitial region(s) of the substrate. Depressions can have any of a variety of shapes at their opening in a surface including, as examples, round, elliptical, square, polygonal, star shaped (with any number of vertices), etc. The cross-section of a depression taken orthogonally with the surface can be curved, square, polygonal, hyperbolic, conical, angular, etc.

The term “each,” when used in reference to a collection of items, is intended to identify an individual item in the collection, but does not necessarily refer to every item in the collection. Exceptions can occur if explicit disclosure or context clearly dictates otherwise.

The term “epoxy” (also referred to as a glycidyl or oxirane group) as used herein refers to

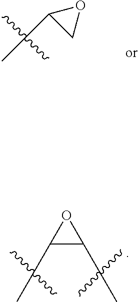

As used herein, the term “flow cell” is intended to mean a vessel having an enclosed or open flow channel where a reaction can be carried out. A flow cell with an enclosed channel also includes an inlet for delivering reagent(s) to the flow channel and an outlet for removing reagent(s) from the flow channel. In some examples, the flow cell enables the detection of the reaction that occurs therein. For example, the flow cell can include one or more transparent surfaces allowing for the optical detection of arrays, optically labeled molecules, or the like at designated reactive regions. As another example, the flow cell can include optics and electronics that facilitate the electrical detection of the reaction that occurs therein.

As used herein, a “flow channel” or “channel” may be (i) an area defined between two bonded components, or maybe (ii) a concave area, or lane, defined in a single substrate. In either case, the “flow channel” or “channel” can selectively receive a liquid sample, reagents, etc. In some examples, the flow channel may be defined between two patterned structures, and thus the flow channel may be in fluid communication with surface chemistry of each of the patterned structures. In other examples, the flow channel may be defined between a patterned structure and a lid, and thus the flow channel may be in fluid communication with surface chemistry of the one patterned structure. In still other examples, the flow channel may be defined by a concave area that is formed in a substrate surface, and thus the flow channel may be in fluid communication with surface chemistry within the concave area.

As used herein, the terms “gas-dissolvable layer,” “gas-dissolvable coating,” and “gas-dissolvable protective coating” refer to a (protective) coating that is capable of undergoing a change in solubility when exposed to a reactive gas. The terms may refer to materials that are capable of preventing the reactive entity from chemically reacting prior to the removal of the gas-dissolvable layer. In some examples disclosed herein, after the gas-dissolvable coating is exposed to the reactive gas, the coating becomes washable using an aqueous solvent.

As used herein, the term “gas-generative species” is a material that is capable of producing, or of being converted to, a reactive gas upon exposure to a predetermined change in temperature or upon exposure to an acid or another pH reducer (resulting in a drop in pH). The reactive gas that is generated may be used to remove a gas-dissolvable protective coating (as defined herein). In some examples described herein, the gas-generative species is included in depressions that are defined in a flow cell substrate.

As used herein, the terms “heat-responsive layer,” “heat-responsive coating,” and “heat-responsive protective coating” refer to a (protective) coating (within a depression or overlying a protrusion, as defined herein) that is capable of undergoing a phase transition (e.g., melting, transitioning from a hydrophobic state to hydrophilic state, transitioning from a hydrophilic state to hydrophobic state, etc.) when exposed to a certain change in temperature. The change in temperature may be generated by a heating mechanism included in a complementary metal oxide semiconductor chip coupled to a flow cell substrate (as described herein), or by a heating mechanism included as part of a lid of the flow cell, or by a heating mechanism that is included/embedded in the flow cell substrate, or by a heating mechanism that is deposited within depressions defined in the flow cell substrate.

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen, and/or sulfur, in the ring backbone. When the heteroaryl is a ring system, every ring in the system is aromatic. The heteroaryl group may have 5-18 ring members.

As used herein, “heterocycle” means a non-aromatic cyclic ring or ring system containing at least one heteroatom in the ring backbone. Heterocycles may be joined together in a fused, bridged or spiro-connected fashion. Heterocycles may have any degree of saturation provided that at least one ring in the ring system is not aromatic. In the ring system, the heteroatom(s) may be present in either a non-aromatic or aromatic ring. The heterocycle group may have 3 to 20 ring members (i.e., the number of atoms making up the ring backbone, including carbon atoms and heteroatoms). In some examples, the heteroatom(s) are O, N, or S.

The term “hydrazine” or “hydrazinyl” as used herein refers to a —NHNH₂ group.

As used herein, the term “hydrazone” or “hydrazonyl” refers to a

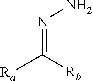

group in which Rₐ and R_{b} are each independently selected from hydrogen, C1-6 alkyl, C2-6 alkenyl, C2-6 alkynyl, C3-7 carbocycle, C6-10 aryl, 5-10 membered heteroaryl, and 5-10 membered heterocycle, as defined herein.

As used herein, “hydroxy” or “hydroxyl” refers to an —OH group.

As used herein, the term “interstitial region” refers to an area, e.g., of a substrate, that separates depressions or protrusions. For example, an interstitial region can separate one depression or protrusion of an array from another depression or protrusion of the array. The two depressions or protrusions that are separated from each other can be discrete, i.e., lacking physical contact with each other. In many examples, the interstitial region is continuous, whereas the depressions or protrusions are discrete, for example, as is the case for a plurality of depressions defined in an otherwise continuous surface or a plurality of protrusions defined on an otherwise continuous surface. Interstitial regions may have a surface material that differs from the surface material of the depressions or protrusions. For example, depressions can have a polymeric hydrogel layer and primers therein, and the interstitial regions can be free of the polymeric hydrogel layer and primers.

“Nitrile oxide,” as used herein, means a “RₐC≡N⁺O⁻” group in which Rₐ is defined herein. Examples of preparing nitrile oxide include in situ generation from aldoximes by treatment with chloramide-T or through action of base on imidoyl chlorides [RC(Cl)═NOH] or from the reaction between hydroxylamine and an aldehyde.

“Nitrone,” as used herein, means a

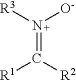

group in which R¹, R², and R³ may be any of the Rₐ and R_{b} groups defined herein, except that R³ is not hydrogen (H).

As used herein, a “nucleotide” includes a nitrogen-containing heterocyclic base, a sugar, and one or more phosphate groups. Nucleotides are monomeric units of a nucleic acid sequence. In RNA, the sugar is a ribose, and in DNA, the sugar is a deoxyribose, i.e. a sugar lacking a hydroxyl group that is present at the 2′ position in ribose. The nitrogen containing heterocyclic base (i.e., nucleobase) can be a purine base or a pyrimidine base. Purine bases include adenine (A) and guanine (G), and modified derivatives or analogs thereof. Pyrimidine bases include cytosine (C), thymine (T), and uracil (U), and modified derivatives or analogs thereof. The C-1 atom of deoxyribose is bonded to N-1 of a pyrimidine or N-9 of a purine. A nucleic acid analog may have any of the phosphate backbone, the sugar, or the nucleobase altered. Examples of nucleic acid analogs include, for example, universal bases or phosphate-sugar backbone analogs, such as peptide nucleic acid (PNA).

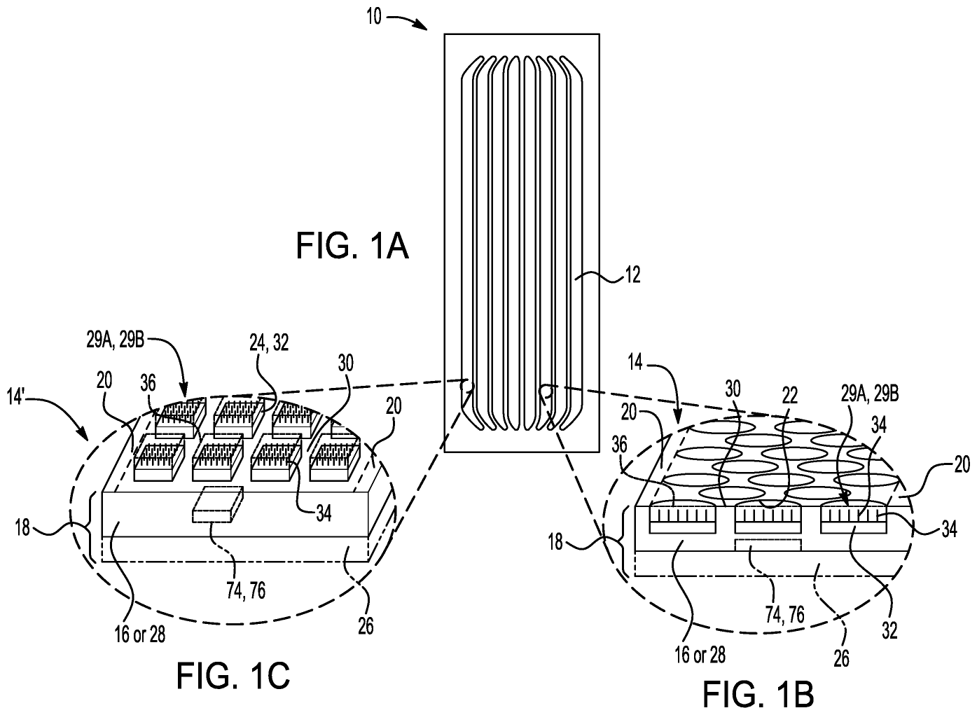

In some examples, the term “over” may mean that one component or material is positioned directly on another component or material. When one is directly on another, the two are in contact with each other. For example, in FIG. 1B, when the multi-layer substrate 18 is used, the layer 28 is positioned directly over the base support 26.

In other examples, the term “over” may mean that one component or material is positioned indirectly on another component or material. By indirectly on, it is meant that a gap or an additional component or material may be positioned between the two components or materials. For example, in FIG. 1B, when the multi-layer substrate 18 is used, the polymeric hydrogel 32 is positioned indirectly over the base support 26. The layer 28 is positioned therebetween.

A “patterned structure” refers to a substrate that includes surface chemistry in a pattern, e.g., in depressions or as protrusions, across the substrate. The surface chemistry may include a polymeric hydrogel layer and primers (e.g., used for library template capture and amplification). In some examples, the substrate has been exposed to patterning techniques (e.g., etching, lithography, etc.) in order to generate the pattern for the surface chemistry. However, the term “patterned structure” is not intended to imply that such patterning techniques have to be used to generate the pattern. The patterned structure may be generated via any of the methods disclosed herein.

As used herein, the term “polyhedral oligomeric silsesquioxane” refers to a chemical composition that is a hybrid intermediate (e.g., RSiO_{1.5}) between that of silica (SiO₂) and silicone (R₂SiO). An example of polyhedral oligomeric silsesquioxane may be that described in Kehagias et al., Microelectronic Engineering 86 (2009), pp. 776-78, which is incorporated by reference in its entirety. In an example, the composition is an organosilicon compound with the chemical formula [RSiO_{3/2}]ₙ, where the R groups can be the same or different. Example R groups for polyhedral oligomeric silsesquioxane include epoxy, azide/azido, a thiol, a poly(ethylene glycol), a norbornene, a tetrazine, acrylates, and/or methacrylates, or further, for example, alkyl, aryl, alkoxy, and/or haloalkyl groups.

As used herein, a “polymeric hydrogel” refers to a gel material that is applied over at least a portion of a substrate. The gel material includes functional group(s) that can attach to a reactive entity, such as primers of a primer set. The polymeric hydrogel layer may be positioned within a portion of a depression defined in the substrate, or may define a protrusion on a substrate.

As used herein, the “primer” is defined as a single stranded nucleic acid sequence (e.g., single strand DNA). Some primers, referred to herein as amplification primers, serve as a starting point for template amplification and cluster generation. Other primers, referred to herein as sequencing primers, serve as a starting point for DNA synthesis. The 5′ terminus of the primer may be modified to allow a coupling reaction with a functional group of a polymer. The primer length can be any number of bases long and can include a variety of non-natural nucleotides. In an example, the sequencing primer is a short strand, ranging from 10 to 60 bases, or from 20 to 40 bases.

As used herein, the term “protrusion” refers to a discrete convex feature defined on a substrate and surrounded by interstitial region(s) of the substrate. Protrusions can have any of a variety of shapes at their opening in a surface including, as examples, round, elliptical, square, polygonal, star shaped (with any number of vertices), etc. The cross-section of a protrusion taken orthogonally with the surface can be curved, square, polygonal, hyperbolic, conical, angular, etc.

As used herein, the term “reactive entity” refers to the flow cell surface chemistry that enables a desired interaction or reaction. As examples, the reactive entity may be a primer that serves as a starting point for template amplification and cluster generation or an enzyme tag, such as a transposome complex used in tagmentation, or reactive functional groups, such as dibenzocyclooctyne (DBCO), strained alkynes or azides, or biotin.

A “reactive region,” as used herein, refers to the discrete area on or in the substrate that includes the polymeric hydrogel and the reactive entity.

A “removable coating”, a “protective coating”, a “protective layer,” or an “independently removable coating” is a layer that is positioned over the reactive region and that can be removed from the reactive region without deleteriously affecting the polymeric hydrogel layer of the reactive entity of the reactive region. These terms may refer to independently removable heat-responsive coatings or gas-dissolvable coatings (as each of these terms is defined herein).

A “spacer layer,” as used herein refers to a material that bonds two components together. In some examples, the spacer layer can be a radiation absorbing material that aids in bonding, or can be put into contact with a radiation absorbing material that aids in bonding.

The term “substrate” refers to a single layer structure or a multi-layer structure (including a base support and another layer positioned thereon) upon which the reactive regions are introduced.

A “thiol” functional group refers to —SH.

As used herein, the terms “tetrazine” and “tetrazinyl” refer to six-membered heteroaryl group comprising four nitrogen atoms. Tetrazine can be optionally substituted.

“Tetrazole,” as used herein, refer to five-membered heterocyclic group including four nitrogen atoms. Tetrazole can be optionally substituted.

### Flow Cells

Some examples of the flow cells disclosed herein include i) a substrate; ii) a plurality of reactive regions spatially separated from one another across the substrate, each of the plurality of reactive regions including: a polymeric hydrogel layer and a reactive entity attached to the polymeric hydrogel layer; and iii) a plurality of independently removable coatings respectively positioned over each of the plurality of reactive regions, wherein at least one of the plurality of independently removable coatings is a gas-dissolvable coating.

Other examples of the flow cells disclosed herein include i) a substrate; ii) a plurality of reactive regions spatially separated from one another across the substrate, each of the plurality of reactive regions including: a polymeric hydrogel layer and a reactive entity attached to the polymeric hydrogel layer; iii) a heating mechanism aligned with at least one of the plurality of reactive regions; and iv) a plurality of independently removable coatings respectively positioned over each of the plurality of reactive regions, wherein at least one of the plurality of independently removable coatings is a heat-responsive coating.

A top view of a flow cell 10 is shown in FIG. 1A, and two different examples of architectures within a flow channel 12 of the flow cell 10 are shown in FIG. 1B and FIG. 1C. While not shown in FIG. 1A through FIG. 1C, an enclosed version of the flow cell 10 may include one patterned structure 14, 14′ bonded to a lid, or two patterned structures 14 or 14′ bonded together. The examples depicted in FIG. 1B and FIG. 1C are open-wafer versions of the flow cell 10 including a single patterned structure 14 or 14′ that is open to a surrounding environment.

The example flow cell 10 shown in FIG. 1A includes eight flow channels 12. While eight flow channels 12 are shown, it is to be understood that any number of flow channels 12 may be included in the flow cell 10 (e.g., a single flow channel 12, four flow channels 12, etc.). When multiple flow channels 12 are included, each flow channel 12 may be isolated from each other flow channel 12 so that fluid introduced into a flow channel 12 does not flow into adjacent flow channel(s) 12.

Each flow channel 12 may include an inlet and an outlet (not shown). The inlet and outlet of each flow channel 12 may be positioned at opposed ends of the flow cell 10. The inlets and outlets of the respective flow channels 12 may alternatively be positioned anywhere along the length and width of the flow channel 12 that enables desirable fluid flow.

The inlet allows fluids to be introduced into the flow channel 12, and the outlet allows fluid to be extracted from the flow channel 12. Each of the inlets and outlets is connected to a fluidic control system (including, e.g., reservoirs, pumps, valves, waste containers, and the like), which controls fluid introduction and expulsion. Some examples of the fluids introduced into the flow channel 12 may introduce reaction components (e.g., DNA sample(s), polymerases, sequencing primers, nucleotides, etc.), washing solutions, deblocking agents, etc.

The flow channel 12 may have any desirable shape. In an example, the flow channel 12 has a substantially rectangular configuration with curved ends (as shown in FIG. 1A). The length of the flow channel 12 depends, in part, upon the size of the substrate (e.g., 16 or 18, see FIG. 1B and FIG. 1C) used to form the patterned structure 14, 14′. The width of the flow channel 12 depends, in part, upon the size of the substrate 16 or 18 used to form the patterned structure 14, 14′, the desired number of flow channels 12, the desired space between adjacent channels 12, and the desired space at a perimeter of the patterned structure 14, 14′. The spaces between flow channels 12 and at the perimeter may be sufficient for attaching the patterned structure 14, 14′ to a lid (not shown in FIG. 1A through FIG. 1C) or to another patterned structure (also not shown).

The flow channel(s) 12 in enclosed versions of the flow cell 10 is/are defined between the one patterned structure 14, 14′ and the lid or between the first patterned structure 14, 14′ and the second patterned structure, which are bonded together via a spacer layer (not shown). Thus, the flow channel(s) 12 in the enclosed form of the flow cell 10 is/are defined by the (i) patterned structure 14, 14′ (ii) the spacer layer, and (iii) either the lid or the second patterned structure. Alternatively, when a single patterned structure 14 is used (e.g., as an open-wafer substrate), the flow channel 12 may be defined by a lane (not shown) that has been patterned into the substrate 14 (e.g., via nanolithography) and in which depressions or protrusions are defined.

The depth of the flow channel 12 in the enclosed versions of the flow cell 10 can be as small as a monolayer thick when microcontact, aerosol, or inkjet printing is used to deposit a separate material (e.g., the spacer layer (not shown)) that defines at least a portion of the sidewalls of the flow channel 12. This depth could be thicker if the spacer layer is pre-formed or applied via another technique. The depth of the flow channel 12 in the open-wafer versions of the flow cell 10 is approximately equivalent to the depth of the lane (although it is deeper in regions where the depressions are formed). In any examples disclosed herein, the depth of the flow channel 12 can be about 1 μm, about 10 μm, about 50 μm, about 100 μm, or more. In an example, the depth may range from about 10 μm to about 400 μm. In another example, the depth may range from about 10 μm to about 30 μm. In still another example, the depth is about 5 μm or less. It is to be understood that the depth of the flow channel 12 may be greater than, less than or between the values specified above,

The spacer layer used to attach the patterned structure 14, 14′ and the lid (or to attach the first patterned structure 14, 14′ and the second patterned structure) may be any material that will seal portions of the patterned structure 14, 14′ and the lid or that will seal portions of the two patterned structures together. As examples, the spacer layer may be an adhesive, a radiation-absorbing material that aids in bonding, or the like. In some examples, the spacer layer is the radiation-absorbing material, e.g., KAPTON® black.

The patterned structure 14, 14′ and the lid (or the first patterned structure 14, 14′ and the second patterned structure) may be bonded using any suitable technique, such as laser bonding, diffusion bonding, anodic bonding, eutectic bonding, plasma activation bonding, glass frit bonding, or others methods known in the art.

When used, the lid may be any material that is transparent to the excitation light that is directed toward the flow cell 10. In optical detection systems, the lid may also be transparent to the emissions generated from reaction(s) taking place in the flow cell 10. As examples, the lid may include glass (e.g., borosilicate, fused silica, etc.) or a transparent polymer. A commercially available example of a suitable borosilicate glass is D 263®, available from Schott North America Inc. Commercially available examples of suitable polymer materials, namely cyclo olefin polymers, are the ZEONOR® products available from Zeon Chemicals L.P. In some instances, the lid is shaped to form the top of the flow cell 10, and in other instances, the lid is shaped to form both the top of the flow cell as well as sidewalls of the flow channel 12. The lid, when used, may include heating mechanism materials (e.g., electrode materials) that align with reactive regions 29A, 29B of the flow cell 10. Examples of suitable electrode materials are described in more detail herein.

The patterned structure 14, 14′ may include a bonding region 20 where it can be sealed to the lid or to the second patterned structure. The bonding region 20 may be located at the perimeter of each flow channel 12 (as shown in FIG. 1B and FIG. 1C) and at the perimeter of the flow cell 10. In the open-wafer version of the flow cell 10, this region 20 outlines the perimeter of the lane.

The patterned structure 14, 14′ includes a substrate 16 or 18, as shown in FIG. 1B and FIG. 1C. The substrate 16 is a single layer structure, and the substrate 18 is a multi-layer structure including a base support 26 and a layer 28 positioned on the base support 26. The substrate 16 may include a single material that has depressions 22 defined therein or protrusions 24 defined thereon. The substrate 18 includes the base support 26 and the layer 28 positioned on the base support 26, where the other layer 28 has the depressions 22 defined therein or the protrusions 24 defined thereon.

Examples of suitable materials for the substrate 16 include siloxanes (e.g., epoxy siloxane), glass, modified or functionalized glass, polymeric materials (including acrylics, polystyrene and copolymers of styrene and other materials, polypropylene, polyethylene, polybutylene, polyurethanes, polytetrafluoroethylene (such as TEFLON® from Chemours), polyethylene terephthalate (PET), polycarbonate, cyclic olefins/cyclo-olefin polymers (COP) (such as ZEONOR® from Zeon), polyimides, nylon (polyamides), etc.) ceramics/ceramic oxides, aluminum silicate, silicon and modified silicon (e.g., boron doped p+silicon), silicon nitride (Si₃N₄), carbon, metals, resins, or the like. Examples of suitable inorganic resins include inorganic oxides, such as tantalum pentoxide (e.g., Ta₂O₅) or other tantalum oxide(s) (TaₓO_{y}), aluminum oxide (e.g., Al₂O₃), silica (i.e., silicon dioxide (SiO₂)), fused silica, or silica-based materials, hafnium oxide (e.g., HfO₂), indium tin oxide, titanium dioxide, etc. Examples of suitable polymeric resins include polyhedral oligomeric silsesquioxane based resin (e.g., POSS® from Hybrid Plastics), a non-polyhedral oligomeric silsesquioxane epoxy resin, a poly(ethylene glycol) resin, a polyether resin (e.g., ring opened epoxies), an acrylic resin, an acrylate resin, a methacrylate resin, an amorphous fluoropolymer resin (e.g., CYTOP® from Bellex), and combinations thereof.

As mentioned, examples of the multi-layer structure (i.e., substrate 18) include the base support 26 and at least one other layer 28 thereon. Any example of the material of the single layer substrate 16 may be used as the base support 26. In examples of the flow cell 10 that include the substrate 18, the other layer 28 may be any material that can be etched or imprinted to form the depressions 22. Examples of the layer 28 include inorganic oxides, such as tantalum oxide (e.g., Ta₂O₅), aluminum oxide (e.g., Al₂O₃), silicon dioxide (e.g., SiO₂), or hafnium oxide (e.g., HfO₂), or polymeric resins, such as a polyhedral oligomeric silsesquioxane based resin (e.g., POSS® from Hybrid Plastics), a non-polyhedral oligomeric silsesquioxane epoxy resin, a poly(ethylene glycol) resin, a polyether resin (e.g., ring opened epoxies), an acrylic resin, an acrylate resin, a methacrylate resin, an amorphous fluoropolymer resin (e.g., CYTOP® from Bellex), and combinations thereof.

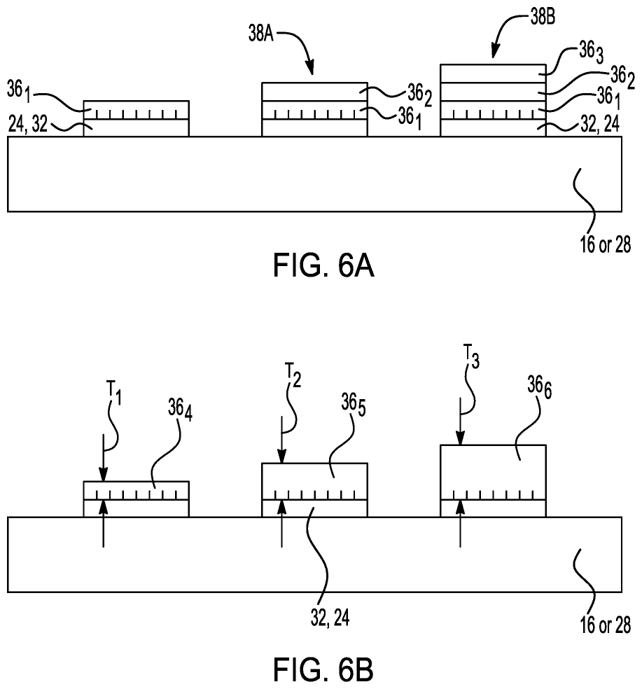

While not shown in FIG. 1A through FIG. 1C, in some examples, the substrate 16 or the layer 28 of the substrate 18 includes heating mechanism materials that align with reactive regions of the substrate 16, 18. An example of a heating mechanism 74 or an electrode 76 is depicted in FIG. 1B and FIG. 1C, where the heating mechanism 74 or the electrode 76 is embedded within the substrate 16, 18. In other examples, the heating mechanism 74 or electrode 76 is positioned at the bottom of each depression 22 (as shown in FIG. 5). In still other examples, the heating mechanism 74 or electrode 76 is affixed to or at least partially embedded in the lid (as shown in phantom in FIG. 5, where the lid is reference numeral 116). As is described in reference to FIG. 5, the heating mechanism 74 is generally used for removal of a heat-responsive coating 36B-1, 36B-2 and the electrode 76 is generally used for removal of a gas-dissolvable coating 36A-1, 36A-2. It is to be understood, however, that some heating mechanism materials may also function as electrodes (i.e., the material is both a heating mechanism and an electrode). It is also to be understood that the materials selected for the electrode 76 may be low resistive materials to minimize heat generation.

When the heating mechanism 74 or electrode 76 is embedded within the substrate 18, a heating mechanism material may be patterned on the base support 26 before the other layer 28 is applied thereon. The pattern of the heating mechanism material will be the same as the pattern for the reactive regions 29A, 29B that are to be formed. When the heating mechanism 74 or electrode 76 is embedded within the substrate 16 or in the lid 116, the heating mechanism material is completely surrounded by the substrate 16 or the lid 116. In these examples, a panel of material suitable for substrate 16 or the lid 116 may be etched or imprinted to form concave regions where the heating mechanism material is introduced, and then additional substrate or lid material may be applied thereon to complete the substrate 16 or lid 116 having the heating mechanism 74 or electrode 76 embedded therein. When the heating mechanism 74 or electrode 76 is partially embedded within the lid 116, the heating mechanism material is partially surrounded by the lid 116. In these examples, a panel of material suitable for the lid 116 may be etched or imprinted to form concave regions where the heating mechanism material is introduced. In these examples, one surface of the heating mechanism 74 or electrode 76 may be exposed at one surface of the lid 116.

In any of the examples set forth herein, the substrate 16 or the base support 26 of the substrate 18 may be a circular sheet, a panel, a wafer, a die, etc. having a diameter ranging from about 2 mm to about 300 mm, e.g., from about 200 mm to about 300 mm, or may be a rectangular sheet, panel, wafer, die etc. having its largest dimension up to about 10 feet (˜3 meters). As one example, a die may have a width ranging from about 0.1 mm to about 10 mm. While example dimensions have been provided, it is to be understood that the substrate 16 or the base support 26 may have any suitable dimensions.

Different examples of the architecture within the flow channel 12 of the flow cell 10 are respectively depicted in FIG. 1B and in FIG. 1C. In FIG. 1B, depressions 22 are defined in the substrate 16 or in the layer 28 of the substrate 18 (to form the patterned structure 14). In FIG. 1C, protrusions 24 are defined on the substrate 16 or on the layer 28 of the substrate 18 (to form the patterned structure 14′).

Many different layouts of the depressions 22 or protrusions 24 may be envisaged, including regular, repeating, and non-regular patterns. In an example, the depressions 22 or protrusions 24 are disposed in a hexagonal grid for close packing and improved density. Other layouts may include, for example, rectangular layouts, triangular layouts, and so forth. In some examples, the layout or pattern can be an x-y format in rows and columns. In some other examples, the layout or pattern can be a repeating arrangement of the depressions 22 or protrusions 24 and interstitial regions 30. In still other examples, the layout or pattern can be a random arrangement of the depressions 22 or protrusions 24 and the interstitial regions 30.

The layout or pattern may be characterized with respect to the density (number) of the depressions 22 or protrusions 24 in a defined area. For example, the depressions 22 or protrusions 24 may be present at a density of approximately 2 million per mm². The density may be tuned to different densities including, for example, a density of about 100 per mm², about 1,000 per mm², about 0.1 million per mm², about 1 million per mm², about 2 million per mm², about 5 million per mm², about 10 million per mm², about 50 million per mm², or more, or less. It is to be further understood that the density can be between one of the lower values and one of the upper values selected from the ranges above, or that other densities (outside of the given ranges) may be used. As examples, a high density array may be characterized as having the depressions 22 or protrusions 24 separated by less than about 100 nm, a medium density array may be characterized as having the depressions 22 or protrusions 24 separated by about 400 nm to about 1 μm, and a low density array may be characterized as having the depressions 22 or protrusions 24 separated by greater than about 1 μm.

The layout or pattern of the depressions 22 or protrusions 24 may also or alternatively be characterized in terms of the average pitch, or the spacing from the center of one depression 22 or protrusion 24 to the center of an adjacent depression 22 or protrusion 24 (center-to-center spacing) or from the right edge of one depression 22 or protrusion 24 to the left edge of an adjacent depression 22 or protrusion 24. The pattern can be regular, such that the coefficient of variation around the average pitch is small, or the pattern can be non-regular in which case the coefficient of variation can be relatively large. In either case, the average pitch can be, for example, about 50 nm, about 0.15 μm, about 0.5 μm, about 1 μm, about 5 μm, about 10 μm, about 100 μm, or more or less. The average pitch for a particular pattern of depressions 22 or protrusions 24 can be between one of the lower values and one of the upper values selected from the ranges herein. In an example, the depressions 22 or protrusions 24 have a pitch (center-to-center spacing) of about 1.5 μm. While example average pitch values have been provided, it is to be understood that other average pitch values may be used.

The size of each depression 22 may be characterized by its volume, opening area, depth, and/or diameter or length and width. For example, the about 0.1 μm³, about 1 μm³, about 10 μm³, or more, or less. For another example, the opening area can range from about 1×10⁻³ μ² to about 100 μm², e.g., about 1×10⁻² μ², about 1 μm², at least about 10 μm², or more, or less. For still another example, the depth can range from about 0.1 μm to about 100 μm, e.g., about 0.5 μm, about 1 μm, about 10 μm, or more, or less. For another example, the depth can range from about 0.1 μm to about 100 μm, e.g., about 0.5 μm, about 1 μm, about 10 μm, or more, or less. For yet another example, the diameter or each of the length and width can range from about 0.1 μm to about 100 μm, e.g., about 0.5 μm, about 1 μm, about 10 μm, or more, or less.

The size of each protrusion 24 may be characterized by its top surface area, height, and/or diameter or length and width. The top surface area can range from about 1×10⁻³ μm² to about 100 μm², e.g., about 1×10⁻² μm², about 0.1 μm², about 1 μm², at least about 10 μm², or more, or less. The height can range from about 0.1 μm to about 100 μm, e.g., about 0.5 μm, about 1 μm, about 10 μm, or more, or less. The diameter or each of the length and the width can range from about 0.1 μm to about 100 μm, e.g., about 0.5 μm, about 1 μm, about 10 μm, or more, or less.

The flow cell 10 also includes the reactive regions 29A and/or 29B. In the example shown in FIG. 1B, the reactive region 29A, 29B includes the polymeric hydrogel 32 applied within the depressions 22 and further includes the reactive entity 34 attached to the polymeric hydrogel 32. In the example shown in FIG. 1C, the reactive region 29A, 29B includes the polymeric hydrogel 32 applied on the substrate 16 or layer 28 in the form of the protrusions 24 and further includes the reactive entity 34 attached to the polymeric hydrogel 32.

The polymeric hydrogel 32 included in the reactive regions 29A, 29B may be any gel material that can swell when liquid is taken up and that can contract when liquid is removed, e.g., by drying. In an example, the gel material is an acrylamide copolymer. Some examples of the acrylamide copolymer are represented by the following structure (I):

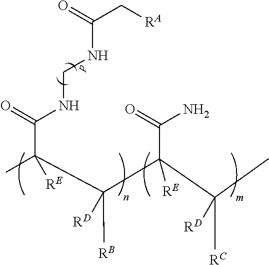

wherein:
R^{A} is selected from the group consisting of azido, optionally substituted amino, optionally substituted alkenyl, optionally substituted alkyne, halogen, optionally substituted hydrazone, optionally substituted hydrazine, carboxyl, hydroxy, optionally substituted tetrazole, optionally substituted tetrazine, nitrile oxide, nitrone, sulfate, and thiol;
R^{B} is H or optionally substituted alkyl;
R^{C}, R^{D}, and R^{E} are each independently selected from the group consisting of H and optionally substituted alkyl;
each of the —(CH₂)ₚ— can be optionally substituted;
p is an integer in the range of 1 to 50;
n is an integer in the range of 1 to 50,000; and
m is an integer in the range of 1 to 100,000.

One specific example of the acrylamide copolymer represented by structure (I) is poly(N-(5-azidoacetamidylpentyl)acrylamide-co-acrylamide), PAZAM.

One of ordinary skill in the art will recognize that the arrangement of the recurring “n” and “m” features in structure (I) are representative, and the monomeric subunits may be present in any order in the polymer structure (e.g., random, block, patterned, or a combination thereof). The molecular weight of the acrylamide copolymer may range from about 5 kDa to about 1500 kDa or from about 10 kDa to about 1000 kDa, or may be, in a specific example, about 312 kDa.

In some examples, the acrylamide copolymer is a linear polymer. In some other examples, the acrylamide copolymer is a lightly cross-linked polymer.

In other examples, the gel material may be a variation of structure (I). In one example, the acrylamide unit may be replaced with N,N-dimethylacrylamide

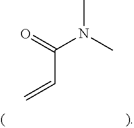

In this example, the acrylamide unit in structure (I) may be replaced with,

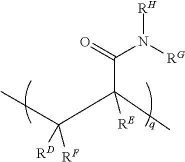

where R^{D}, R^{E}, and R^{F} are each H or a C1-C6 alkyl, and R^{G} and R^{H} are each a C1-C6 alkyl (instead of H as is the case with the acrylamide). In this example, q may be an integer in the range of 1 to 100,000. In another example, the N,N-dimethylacrylamide may be used in addition to the acrylamide unit. In this example, structure (I) may include

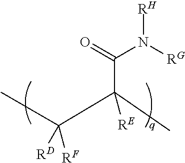

in addition to the recurring “n” and “m” features, where R^{D}, R^{E}, and R^{F} are each H or a C1-C6 alkyl, and R^{G} and R^{H} are each a C1-C6 alkyl. In this example, q may be an integer in the range of 1 to 100,000.

As another example of the gel material, the recurring “n” feature in structure (I) may be replaced with a monomer including a heterocyclic azido group having structure (II):

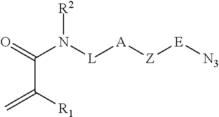

wherein R¹ is H or a C1-C6 alkyl; R₂ is H or a C1-C6 alkyl; L is a linker including a linear chain with 2 to 20 atoms selected from the group consisting of carbon, oxygen, and nitrogen and 10 optional substituents on the carbon and any nitrogen atoms in the chain; E is a linear chain including 1 to 4 atoms selected from the group consisting of carbon, oxygen and nitrogen, and optional substituents on the carbon and any nitrogen atoms in the chain; A is an N substituted amide with an H or a C1-C4 alkyl attached to the N; and Z is a nitrogen containing heterocycle. Examples of Z include 5 to 10 carbon-containing ring members present as a single cyclic structure or a fused structure. Some specific examples of Z include pyrrolidinyl, pyridinyl, or pyrimidinyl.

As still another example, the gel material may include a recurring unit of each of structure (III) and (IV):

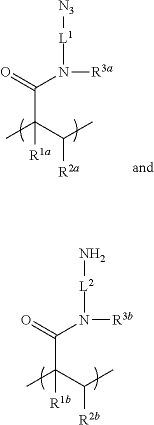

wherein each of R^{1a}, R^{2a}, R^{1b} and R^{2b} is independently selected from hydrogen, an optionally substituted alkyl or optionally substituted phenyl; each of R^{3b} and R^{3b} is independently selected from hydrogen, an optionally substituted alkyl, an optionally substituted phenyl, or an optionally substituted C7-C14 aralkyl; and each L¹ and L² is independently selected from an optionally substituted alkylene linker or an optionally substituted heteroalkylene linker.

In further examples, the polymeric hydrogel 32 is an alginate, acrylamide, or a PEG based material disclosed herein. In some examples, the polymeric hydrogel 32 is a PEG-based material with acrylate-dithiol, or epoxide-amine reaction chemistries. In some examples, the polymeric hydrogel 32 includes PEG-maleimide/dithiol oil, PEG-epoxide/amine oil, PEG-epoxide/PEG-amine, or PEG-dithiol/PEG-acrylate.

Still further examples of suitable polymeric materials for the hydrogel 32 include functionalized polysilanes, such as norbornene silane, azido silane, alkyne functionalized silane, amine functionalized silane, maleimide silane, or any other polysilane having functional groups that can attach the reactive entity 34. Other examples of suitable hydrogel materials for the hydrogel 32 include those having a colloidal structure, such as agarose; or a polymer mesh structure, such as gelatin; or a cross-linked polymer structure, such as polyacrylamide polymers and copolymers, silane free acrylamide (SFA), or an azidolyzed version of SFA. Examples of suitable polyacrylamide polymers may be synthesized from acrylamide and an acrylic acid or an acrylic acid containing a vinyl group, or from monomers that form [2+2] photo-cycloaddition reactions. Still other examples of suitable polymeric hydrogel materials include mixed copolymers of acrylamides and acrylates. A variety of polymer architectures containing acrylic monomers (e.g., acrylamides, acrylates etc.) may be utilized in the examples disclosed herein, such as highly branched polymers, including dendrimers. For example, the monomers (e.g., acrylamide, etc.) may be incorporated, either randomly or in block, into the branches (arms) of a dendrimer.

An example of the dendritic polymeric hydrogel material includes a dendritic core with recurring units of formulas (III) and (IV) in the arms extending from the dendritic core. The dendritic core may have anywhere from 3 arms to 30 arms.

The dendritic core may be any multi-functional component that enables a controlled polymerization mechanism, which leads to a defined arm length in the polymer structure and an at least substantially uniform arm length between polymer structures. In an example, the arms of the dendritic core are identical to each other.

The central molecule/compound of the dendritic core may be any multi-functional molecule, such as macrocycles (e.g., cyclodextrins, porphyrins, etc.), extended pi-systems (e.g., perylenes, fullerenes, etc.), metal-ligand complexes, polymeric cores, etc. Some specific examples of the central molecule/compound of the dendritic core include a phenyl group, benzoic acid, pentraerythritol, a phosphazene group, etc.

The dendritic core includes arms that extend from the central molecule/compound. Each arm may include a group that enables the monomers of formula (III) and (IV) to be incorporated. In one example, a thiocarbonylthio group is included in each arm, and thus includes a reversible addition-fragmentation chain transfer agent (a RAFT agent). In another example, the dendritic core includes an atom transfer radical polymerization (ATRP) initiator in each arm. In still another example, the dendritic core includes a nitroxide (aminooxyl) mediated polymerization (NMP) initiator in each arm.

Functional groups in one or more of the recurring units of the hydrogel material of the hydrogel 32 are capable of attaching the reactive entity 34. These functional groups (e.g., R² in formula (I), NH₂, N₃, etc.) may be located in the side chains of the linear or branched polymeric hydrogel material. As noted, one example of the branched polymeric hydrogel material is a dendrimer, and in an example, the primer-grafting functional groups are located in each of the arms of the dendrimer. These functional groups may be introduced as part of the monomer(s) used in copolymerization. To control the number of reactive entity 34 anchorage points, the monomer bearing the functional group may be increased or decreased. These functional groups may alternatively be introduced after copolymerization.

It is to be understood that other molecules may be used to form the polymeric hydrogel 32, as long as they are capable of being functionalized with the desired chemistry, e.g., of grafting the reactive entity 34 thereto.

The gel material for the polymeric hydrogel 32 may be formed using any suitable copolymerization process, such as nitroxide mediated polymerization (NMP), reversible addition-fragmentation chain-transfer (RAFT) polymerization, etc.

The attachment of the polymeric hydrogel 32 to the underlying component (e.g., substrate 16 or layer 28) may be through covalent bonding. In some instances, the underlying substrate 16 or layer 28 may first be activated, e.g., through silanization or plasma ashing, prior to attaching the polymeric hydrogel 32 thereto. Covalent linking is helpful for maintaining the polymeric hydrogel 32 (and thus the reactive entity 34) in the desired regions throughout the lifetime of the flow cell 10 during a variety of uses.

In each example, the polymeric hydrogel 32 has a reactive entity 34 attached thereto. In some examples of the flow cell 10, the reactive entity 34 in each of the plurality of reactive regions 29A, 29B is a primer set. In one of these examples, the primer set is the same in each of the plurality of reactive regions 29A, 29B. In other of these examples, the primer set of at least one of the plurality of reactive regions 29A, 29B is different than the primer set of at least one other of the plurality of reactive regions 29B, 29A.

The primer set that may be used as the reactive entity 34 includes two different primers that are used in sequential paired-end sequencing. In another example, the reactive entity 34 is an enzyme tag, such as a transposome complex.

As such, in one example, the reactive entity 34 in each of the plurality of reactive regions 29A, 29B is independently selected from the group consisting of a primer set and an enzyme tag.

As mentioned, the reactive entity 34 may be a primer set. The primer set, when used, includes two different primers that are used in sequential paired end sequencing. As examples, the primer set may include P5 and P7 primers, P15 and P7 primers, or any combination of the PA primers, the PB primers, the PC primers, and the PD primers set forth herein. As examples, the primer set may include any two PA, PB, PC, and PD primers, or any combination of one PA primer and one PB, PC, or PD primer, or any combination of one PB primer and one PC or PD primer, or any combination of one PC primer and one PD primer.

Examples of P5 and P7 primers are used on the surface of commercial flow cells sold by Illumina Inc. for sequencing, for example, on HISEQ™, HISEQX™, MISEQ™, MISEQDX™, MINISEQ™, NEXTSEQ™, NEXTSEQDX™, NOVASEQ™, ISEQ™, GENOME ANALYZER™, and other instrument platforms.

The P5 primer (shown as a cleavable primer due to the cleavable nucleobase uracil or “n”) is:

P5 #1: 5′→3′
(SEQ. ID. NO. 1)
AATGATACGGCGACCACCGAGAUCTACAC;
P5 #2: 5′→3′
(SEQ. ID. NO. 2)
AATGATACGGCGACCACCGAGAnCTACAC

where “n” is inosine in SEQ. ID. NO. 2; or

P5 #3: 5′→3′
(SEQ. ID. NO. 3)
AATGATACGGCGACCACCGAGAnCTACAC

where “n” is alkene-thymidine (i.e., alkene-dT) in SEQ. ID. NO. 3.

The P7 primer (shown as cleavable primers) may be any of the following:

P7 #1: 5′→3′
(SEQ. ID. NO. 4)
CAAGCAGAAGACGGCATACGAnAT

where “n” is 8-oxoguanine in SEQ. ID. NO. 4;

P7 #2: 5′→3′
(SEQ. ID. NO. 5)
CAAGCAGAAGACGGCATACnAGAT

where “n” is 8-oxoguanine in SEQ. ID. NO. 5;

P7 #3: 5′→3′
(SEQ. ID. NO. 6)
CAAGCAGAAGACGGCATACnAnAT

where both instances of “n” are 8-oxoguanine in SEQ. ID. NO. 6;

P7 #4: 5′→3′
(SEQ. ID. NO. 7)
CAAGCAGAAGACGGCATACGAUAT;
or
P7 #5: 5′→3′
(SEQ. ID. NO. 8)
CAAGCAGAAGACGGCATACUAGAT.

The P15 primer (shown as a cleavable primer) is:

P15: 5′→3′
(SEQ. ID. NO. 9)
AATGATACGGCGACCACCGAGAnCTACAC

where “n” is allyl-T (i.e., a thymine nucleotide analog having an allyl functionality).

The other primers (PA-PD, shown as non-cleavable primers) mentioned above include:

PA 5′→3′
(SEQ. ID. NO. 10)
GCTGGCACGTCCGAACGCTTCGTTAATCCGTTGAG
PB 5′→3′
(SEQ. ID. NO. 11)
CGTCGTCTGCCATGGCGCTTCGGTGGATATGAACT
PC 5′→3′
(SEQ. ID. NO. 12)
ACGGCCGCTAATATCAACGCGTCGAATCCGCAACT,
and
PD 5′→3′
(SEQ. ID. NO. 13)
GCCGCGTTACGTTAGCCGGACTATTCGATGCAGC.

Complementary versions of the example PA-PD primers set forth herein may also be used. While not shown in the example sequences for PA-PD, it is to be understood that any of these primers may include a cleavage site, such as uracil, 8-oxoguanine, allyl-T, etc. at any point in the strand.

Each of the primers disclosed herein may also include a polyT sequence at the 5′ end of the primer sequence. In some examples, the polyT region includes from 2 T bases to 20 T bases. As specific examples, the polyT region may include 3, 4, 5, 6, 7, or 10 T bases.

The 5′ end of each primer may also include a linker. Any linker that includes a terminal alkyne group, an internal alkyne group, or another suitable terminal functional group that can attach to the surface functional groups of the polymeric hydrogel 32 may be used. In one example, the primers are terminated with hexynyl functional groups. In another example, the 5′ end functional groups of the primers include an internal alkyne, where the alkyne is part of a cyclic compound (e.g., bicyclo[6.1.0]nonyne (BCN) or dibenzocyclooctyne (DBCO)).

As described, in some examples, the same reactive entity 34 (e.g., primer set) is attached to the polymeric hydrogel 32 that is present in each of the depressions 22 or that forms each of the protrusions 24. For example, the primer set may be the same in each of the plurality of reactive regions 29A, 29B, when the primer set is used as the reactive entity 34. In other examples, one reactive entity 34 (e.g., a primer set including P5 and P7 primers) is attached in a subset of the depressions 22 or to a subset of the protrusions 24, while a different reactive entity 34 (e.g., another primer set including PA and PB primers) is attached in a subset of the depressions 22 or to a subset of the protrusions 24. As such, in some examples (and as described herein), the primer set of at least one of the plurality of reactive regions 29A, 29B is different than the primer set of at least one other of the plurality of reactive regions 29A, 29B.

The architecture shown in FIG. 1B may be generated by forming the depressions 22 in the substrate 16 or the layer 28 of the substrate 18, introducing the polymeric hydrogel 32 into the depressions 22, and attaching the reactive entity 34 (e.g., primers of a primer set) the polymeric hydrogel 32.

The depressions 22 may be formed (in the substrate 16 or the layer 28 of the substrate 18) using etching or nanoimprint lithography.

A mixture of the polymeric hydrogel 32 may be generated. In one example, the polymeric hydrogel 32 may be present in a mixture (e.g., with water or with ethanol and water). The polymeric hydrogel 32 may be blanketly deposited over the substrate 16 or over the layer 28 of the substrate 18, and then removed from the interstitial regions 30 using a polishing technique.

The reactive entity 34 may then be attached to the polymeric hydrogel 32. As an example, primers may be grafted to the polymeric hydrogel 32. Grafting may be accomplished by flow through deposition (e.g., using a temporarily bound lid), dunk coating, spray coating, puddle dispensing, or by another suitable method. Each of these example techniques may utilize a reactive entity solution or mixture, which may include the reactive entity 34, water, a buffer, and a catalyst. With any of the grafting methods, the reactive entity 34 attaches to the reactive groups of the polymeric hydrogel 32 and does not react with the interstitial regions 30. As another example, enzyme tags may be attached through oligonucleotide hybridization or through biotin/streptavidin interactions. With oligonucleotide hybridization, the enzyme tag may include an oligonucleotide sequence that can hybridize to polymeric hydrogel bound primers. With biotin/streptavidin interactions, the polymeric hydrogel 32 may be biotinylated, and the enzyme tag may include streptavidin or streptavidin-biotin.

When a single type of reactive entity 34 is used, the reactive entity 34 may be alternatively be pre-grafted to the polymeric hydrogel 32, and the pre-grafted hydrogel may be deposited and polished, or selectively deposited.

When multiple reactive entities 34 are used, some depressions 22 may be masked (e.g., with a photoresist or other suitable mask) while other depressions 22 are grafted with one type of reactive entity 34. Alternatively, the reactive entities 34 may be pre-grafted to different examples of the polymeric hydrogel 32 and the pre-grafted hydrogels may be respectively and selectively deposited into the desired depressions 22.

The architecture shown in FIG. 1C may be generated by forming the protrusions 24 on the substrate 16 or the layer 28 of the substrate 18 using the polymeric hydrogel 32, and attaching the reactive entity 34 to the polymeric hydrogel 32. A mixture of the polymeric hydrogel 32 may be generated as described herein.

In one example, a photoresist may first be deposited on the substrate 16 or the layer 28, and developed such that soluble photoresist portions are removed where it is desirable to form the protrusions 24 and insoluble photoresist portions remain where it is desirable to form the interstitial regions 30. The mixture including the polymeric hydrogel 32 may be blanketly deposited over the insoluble photoresist portions and over the exposed portions of the substrate 16 or the layer 28, and cured. The polymeric hydrogel 32 applied to the exposed portions of the substrate 16 or the layer 28 become the protrusions 24. The insoluble photoresist portions, and the polymeric hydrogel 32 thereon, may be removed using a suitable remover for the photoresist to expose the interstitial regions 30.

Alternatively, the mixture including the polymeric hydrogel 32 may be selectively deposited (using a mask to cover interstitial regions 30, controlled printing techniques, etc.) to specifically deposit the polymeric hydrogel 32 at areas where it is desirable to form the protrusions 24.

The reactive entity 34 may then be grafted to the protrusions 24. As examples, grafting may be accomplished by flow through deposition (e.g., using a temporarily bound lid), dunk coating, spray coating, puddle dispensing, or by another suitable method. Each of these example techniques may utilize a reactive entity 34 solution or mixture, which may include the reactive entity 34, water, a buffer, and/or a catalyst. With any of the grafting methods, the reactive entity 34 attaches to the reactive groups of the polymeric hydrogel 32/protrusions 24 and does not react with the interstitial regions 30.

When a single reactive entity 34 is used, the reactive entity 34 may be pre-grafted to the polymeric hydrogel 32, and the pre-grafted hydrogel may be deposited to form the protrusions 24 according to the examples set forth herein.

When multiple reactive entities 34 are used, some protrusions 24 may be masked (e.g., with a photoresist or other suitable mask) while other protrusions 24 have one type of reactive entity 34 grafted thereto. Alternatively, the reactive entities 34 may be pre-grafted to different samples of polymeric hydrogel 32 and the pre-grafted hydrogels may be respectively and selectively deposited to form the protrusions 24.

As shown in FIG. 1B and FIG. 1C, each of the flow cell architectures also includes the independently removable coating(s) 36 positioned over the reactive regions 29A, 29B (and thus the coating(s) 36 are positioned over the polymeric hydrogel 32 and over the reactive entity 34). The independently removable coating(s) 36 render the reactive regions 29A, 29B inactive (e.g., incapable of participating in a designated chemical reaction) until the independently removable coating(s) 36 overlying the particular reactive region 29A, 29B is/are removed to expose the reactive region 29A, 29B. As such, the coating(s) 36 can be designed so that particular reactive region(s) 29A, 29B is/are exposed for analysis or for a designated reaction at a particular time.

In some examples, the substrate 16, 18 includes a plurality of depressions 22; each of the plurality of reactive regions 29A, 29B is positioned within a respective one of the plurality of depressions 22; and each of the plurality of independently removable coatings 36 covers a respective one of the plurality of reactive regions 29A, 29B. One of these examples is shown in FIG. 1B.

In some other examples, the substrate 16, 18 includes a plurality of protrusions 24; each of the plurality of reactive regions 29A, 29B is positioned at a respective one of the plurality of protrusions 24; and each of the plurality of independently removable coatings 36 covers a respective one of the plurality of protrusions 24. One of these examples is shown in FIG. 1C.

Each of the plurality of independently removable coatings 36 may have a thickness ranging from about 10 nm to about 1000 nm.

Different examples of the materials and removal characteristics of the independently removable coatings 36 will now be described.

In some examples of the flow cell 10, at least one of the plurality of independently removable coatings 36 is the gas-dissolvable coating 36A-1 or 36A-2 (shown in FIG. 5). Methods of using the gas-dissolvable coating 36A-1 or 36A-2 will be described in more detail herein in regard to FIG. 5. The gas-dissolvable coating 36A-1, 36A-2 includes a material that is capable of dissolving or undergoing physical and chemical changes (e.g., in terms of solubility) when exposed to a reactive gas (e.g., carbon dioxide, oxygen, or a reactive oxygen species). As will be described in more detail herein, the gas-dissolvable coating 36A-1, 36A-2 may also be capable of being removed from the reactive regions 29A, 29B via exposure to water (or another suitable aqueous solvent) and an inert gas (e.g., N₂) during a removal process.

Individual gas-dissolvable coatings 36A-1, 36A-2 may be separately positioned over each of the depressions 22 or over each of the protrusions 24, or over sub-sets of depressions 22 or protrusions 24, or one gas-dissolvable coating 36A-1, 36A-2 may be in the form of a single layer that extends over all of the reactive regions 29A, 29B (including over the interstitial regions 30).

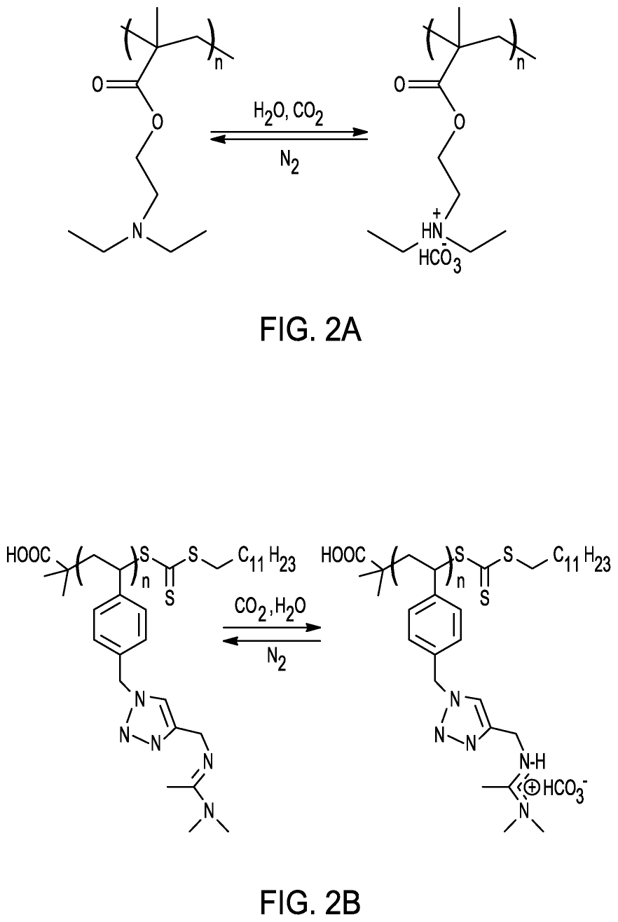

One example of a chemical structure of a suitable gas-dissolvable coating 36A-1, 36A-2 is shown in FIG. 2A. As shown in FIG. 2A, exposure of an amine of the gas-dissolvable coating 36A-1, 36A-2 to water and CO2 gas in the presence of an inert gas (e.g., nitrogen gas (N₂), argon gas, etc.) protonates the nitrogen atom of the gas-dissolvable coating 36A-1, 36A-2, rendering the structure of the coating more hydrophilic (and thus removable using an aqueous solvent). Another example of the chemical structure of a suitable gas-dissolvable coating 36A-1, 36A-2 is shown in FIG. 2B. As shown in FIG. 2B, exposure of the structure to water and CO₂ gas from a gas source in the presence of an inert gas (e.g., N₂) protonates the terminal nitrogen atom outside of the triazole, rendering the structure of the coating more hydrophilic (and thus removable using an aqueous solvent).

Further examples of chemical structures of suitable gas-dissolvable coatings 36A-1, 36A-2 include

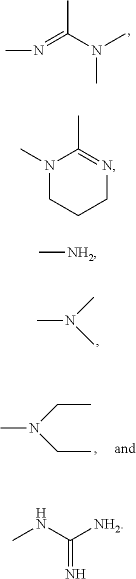

Another example of a suitable gas-dissolvable coating 36A-1, 36A-2 includes a thioether copolymer that transitions from a hydrophobic state to a hydrophilic state (e.g., transitions to a sulfoxide or a sulfone) upon exposure to oxygen or a reactive oxygen species (such as hydrogen peroxide). Still another example of a suitable gas-dissolvable coating 36A-1, 36A-2 includes a phenylboronic acid pinacol ester that transitions from a hydrophobic state to a hydrophilic (e.g., phenolic) state upon exposure to a reactive oxygen species, such as hydrogen peroxide. Yet another example of a suitable gas-dissolvable coating 36A-1, 36A-2 includes a trifluoro ethyl methacrylate copolymer, which becomes transparent upon exposure to oxygen or a reactive oxygen species, and becomes turbid when exposed to N₂ purging.

In examples, the gas-dissolvable coating 36A-1, 36A-2 may be selected from the group consisting of an amine-based coating, an amidine-based coating, a guanidine-based coating, an oxygen-responsive copolymer, an oxidation-responsive copolymer, and combinations thereof.

In some instances, two different gas-dissolvable protective coatings 36A-1, 36A-2 are included in the flow cell 10, where one gas-dissolvable coating 36A-1, 36A-2 covers the reactive region 29A and a different gas-dissolvable coating 36A-2, 36A-1 covers the reactive region 29B. In these examples, the removal condition(s) (e.g., the reactive gas) that is/are used to remove the first gas-dissolvable coating 36A-1, 36A-2 (e.g., overlying the reactive region 29A or 29B) is insufficient to remove the other gas-dissolvable coating 36A-2, 36A-1 (e.g., overlying the reactive region 29B or 29A). In other instances, the same gas-dissolvable coating 36A-1, 36A-2 is used in each of the reactive regions 29A, 29B.

While not shown in FIG. 1B or FIG. 1C (and as described in more detail herein in regard to FIG. 5), when the gas-dissolvable coating 36A-1, 36A-2 is used in the flow cell 10, each of the reactive regions 29A, 29B may include a gas-generative species that is capable of undergoing a chemical reaction that produces a reactive gas. Alternatively, when the gas-dissolvable coating 36A-1, 36A-2 is used in the flow cell 10, each of the reactive regions 29A, 29B may align with an electrode 76 that is capable of producing a reactive gas (e.g., a carbon anode, which generates carbon dioxide gas, or water electrolysis electrodes that produce oxygen gas, such as platinum anodes, nickel anodes, titanium anodes, and the like).

In other examples of the flow cell 10, at least one of the plurality of independently removable coatings 36 is the heat-responsive coating 36B-1 or 36B-2 (also shown in FIG. 5). Methods of using the heat-responsive coating 36B-1, 36B-2 will be described in more detail herein in regard to FIG. 5. Examples of suitable heat-responsive coatings 36B-1, 36B-2 include polymeric materials that are capable of undergoing a phase change upon reaching a certain temperature. As an example, the heat-responsive polymer (of the heat-responsive coating 36B-1, 36B-2) may be a polymer that melts upon reaching a certain temperature. As another example, the heat-responsive polymer may be a polymer that transitions from a hydrophobic state to a hydrophilic state upon reaching a certain temperature, e.g., UCST (upper critical solution temperature) polymers, such as poly(acrylamide-co-acrylonitrile). As yet another example, the heat-responsive polymer may be a polymer that transitions form a hydrophilic state to a hydrophobic state upon reaching a certain temperature, e.g., LCST (lower critical solution temperature) polymers, such as poly(N-isopropylacrylamide) or PNIPAAm, which has a transition temperature of about 32° C.-44° C.

In examples, the heat-responsive coating 36B-1, 36B-2 may include a polymer that is selected from the group consisting of polylactic acid, poly(lactic-co-glycolic) acid, polycaprolactone, agarose, wax, poly(acrylamide-co-acrylonitrile), poly(N-isopropylacrylamide), cyclodextrins, polyethylene glycol homopolymer, polyethylene glycol graft copolymer, polyethylene block copolymer, and a combination thereof. An example of the wax that may be used as the heat-responsive coating 36B-1, 36B-2 includes paraffin, which melts at a temperature ranging from about 53° C. to about 55° C. Another example of the wax that may be used as the heat-responsive coating 36B-1, 36B-2 includes gel-based waxes, some examples of which melt at a temperature ranging from about 75° C. to about 85° C. Additional examples of the wax that may be used as the heat-responsive coating 36B-1, 36B-2 include spermaceti wax (melts at a temperature ranging from about 41° C. to about 49° C.), beeswax (melts at a temperature ranging from about 61° C. to about 63° C., and candelilla wax (melts at a temperature ranging from about 53° C. to about 55° C.).

Further specific examples of suitable materials and some corresponding transition temperatures for the heat-responsive coating 36B-1, 36B-2 include natural polymers, such as gelatin (about 40° C.), hydroxypropylcellulose (45° C.-55° C.), and methylcellulose (about 80° C.). Further examples include synthetic polymers, such as poly(N-isopropylmethacrylamide) (38° C.-44° C.), poly(N,N-diethylacrylamide) (32° C.-34° C.), poly(methylvinylether) (about 37° C.), polyvinyl alcohol (PVA) (about 125° C.), polyvinyl pyrrolidone (PVP) (about 160° C.), poly(methacrylic) acid (about 75° C.), and poly(N-vinyl caprolactam) (about 30° C.-50° C.). Still further examples include co-polymers, such as poly(N-isopropylmethacrylamide)-co-acrylamide (about 33° C.-35° C.), chitosan-graft poly(N-isopropylmethacrylamide)-co-N,N-dimethylacrylamide (about 38° C.), poly(N-isopropylmethacrylamide)-co-N-hydroxymethyl acrylamide (about 34° C.-38° C.), poly(N,N-dimethylaminoethyl) methacrylate-co-ethylene glycol diacrylate (about 32° C.-50° C.), and block copolymers of polyethylene oxide and polypropylene oxide (PEO-b-PPO copolymers) (ranging from 20° C.-85° C.). Yet further examples include polymers modified with magnetic nanoparticles, such as poly(N-isopropylmethacrylamide) embedded with 18 wt % iron oxide nanoparticles (about 32° C.-40° C.) or poly(N-isopropylmethacrylamide) embedded with 38 wt % iron oxide nanoparticles (above 50° C.) Even further examples of suitable materials for the heat-responsive coating 36B-1, 36B-2 and some corresponding LCST temperatures (in degrees Celsius) include PEG-based polymers and copolymers (ranging from 20° C.-85° C.), hydroxypropyl cellulose (about 40° C.-45° C.), hydroxypropylmethyl cellulose (about 69° C.), ethylhydroxyethyl cellulose (about 35° C.), and poly(asparagine) derivatives (about 28° C.-78° C.).

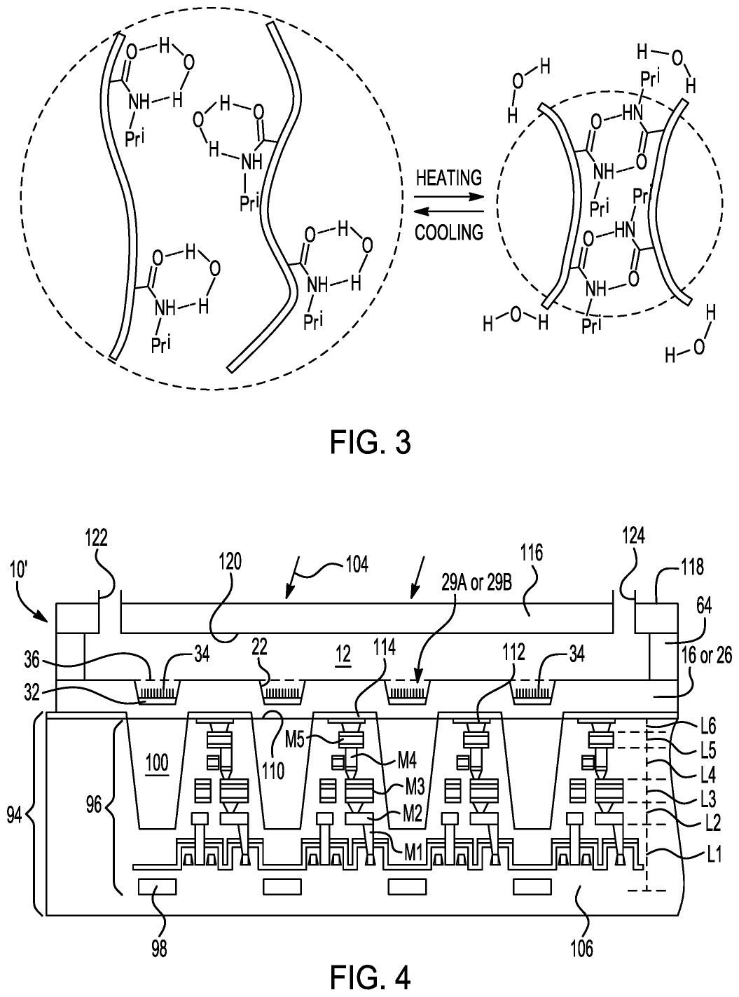

One example of a chemical structure for the heat-responsive coating 36B-1, 36B-2 is shown in FIG. 3, and this chemical structure represents the structure of a PNIPAAm polymer (where “Prⁱ” constituents represent isopropyl groups). As shown in FIG. 3, heating of the polymer results in the restructuring of hydrogen bonds within the polymer's chemical structure, which results in a physical phase change within the polymer that facilitates its removal from the flow cell 10.

Individual heat-responsive coatings 36B-1, 36B-2 may be separately positioned over each of the depressions 22 or over each of the protrusions 24, or one removable heat-responsive coating 36B-1, 36B-2 may be in the form of a single layer that extends over all of the reactive regions 29A, 29B (including over the interstitial regions 30).

In some instances, two different heat-responsive protective coatings 36B-1, 36B-2 are utilized, where one heat-responsive coating 36B-1, 36B-2 covers the reactive region 29A and a different heat-responsive coating 36B-2, 36B-1 covers the reactive region 29B. In these examples, the heating conditions that are used to remove one of the heat-responsive coatings 36B-1, 36B-2 (e.g., overlying the reactive region 29A or 29B) is insufficient to remove the other of the heat-responsive coatings 36B-2, 36B-1 (e.g., overlying the reactive region 29B or 29A). In other instances, the same heat-responsive protective coating 36B-1, 36B-2 is used to cover both reactive regions 29A, 29B.

As is described in more detail herein in regard to FIG. 5, the heat-responsive coating(s) 36B-1, 36B-2 may be capable of being removed using heat that is generated by a heating mechanism 74, where the heating mechanism 74 is included in the lid 116 of the flow cell 10 (when the lid 116 is utilized), or is deposited in depressions 22 defined in the flow cell 10, or is embedded in the substrate 16 or layer 28 of the substrate 18 of the flow cell 10, or as a component of a complementary metal oxide semiconductor chip that is coupled to the substrate 16, 26 of the flow cell 10. Examples of the flow cell including the complementary metal oxide semiconductor chip (e.g., flow cell 10′) will now be described.

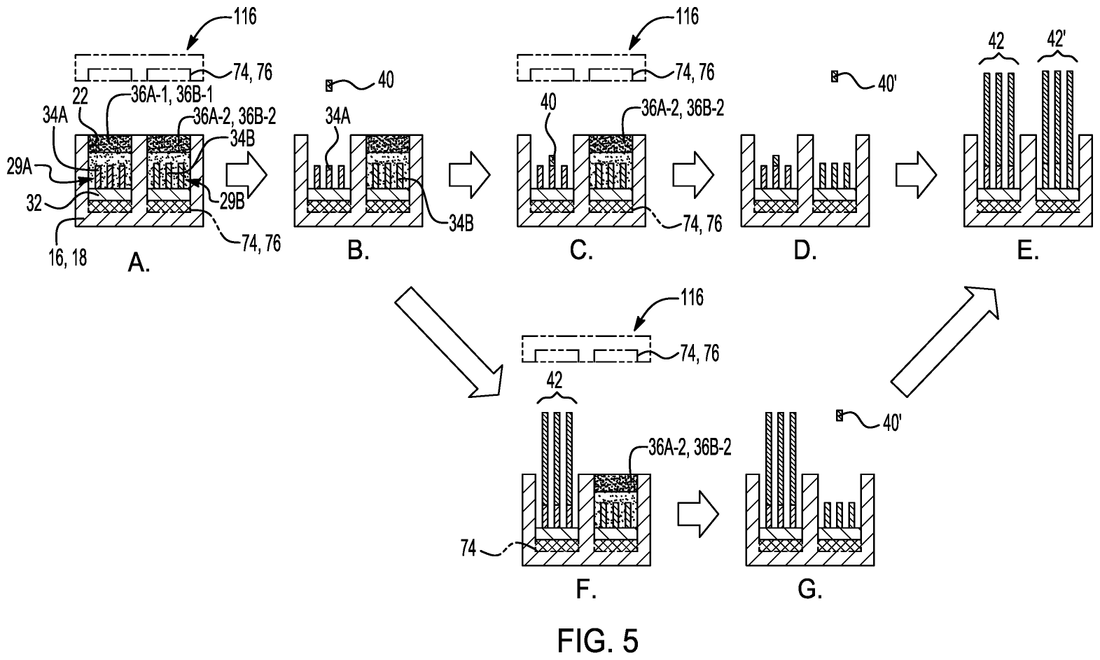

In addition to the components set forth herein for the flow cell 10, the flow cell 10′ further includes a complementary metal oxide semiconductor (CMOS) chip 94 that is attached to a bottom of the substrate 16 or that is attached to a bottom of the base support 26 (e.g., of the substrate 18). This flow cell 10′ is depicted in FIG. 4.

In addition to the CMOS chip 94, this example flow cell 10′ includes i) the substrate 16 or 26; ii) the plurality of reactive regions 29A, 29B spatially separated from one another across the substrate 16 or 26, each of the plurality of reactive regions including: a polymeric hydrogel layer 32, and a reactive entity 34 attached to the polymeric hydrogel layer 32; iii) the heating mechanism 74 (not shown in FIG. 4) aligned with at least one of the plurality of reactive regions 29A, 29B; and iv) a plurality of independently removable coatings 36 respectively positioned over each of the plurality of reactive regions 29A, 29B, wherein at least one of the plurality of independently removable coatings 36 is the heat-responsive coating 36B-1, 36B-2 described herein. It is to be understood that examples of the gas-dissolvable coating 36A-1, 36A-2 described herein may be used as the removable coating(s) 36 for the flow cell 10′. In these examples, the heating mechanism 74 may be replaced with the electrode 76 that is capable of generating the reactive gas.

The flow cell 10′ includes the reactive entity 34 within the reactive region(s) 29A, 29B. The reactive entity 34 in each of the plurality of reactive regions 29A, 29B may be a primer set. In some examples, the primer set is the same in each of the plurality of reactive regions 29A, 29B. In other examples, the primer set of at least one of the plurality of reactive regions 29A, 29B is different than the primer set of at least one of the other of the plurality of reactive regions 29B, 29A.

In some examples, the substrate 16, 26 of the flow cell 10′ includes a plurality of depressions 22, each of the plurality of reactive regions 29A, 29B is positioned within a respective one of the plurality of depressions 22, and each of the plurality of independently removable coatings 36 covers a respective one of the plurality of reactive regions 29A, 29B. This is shown in FIG. 4.

While not shown in FIG. 4, in some other examples of the flow cell 10′, the substrate 16, 26 includes a plurality of protrusions 24, each of the plurality of reactive regions 29A, 29B is positioned at a respective one of the plurality of protrusions 24, and each of the plurality of independently removable coatings 36 covers a respective one of the plurality of protrusions 24.

Each of the plurality of independently removable coatings 36 may be the gas-dissolvable coating 36A-1, 36A-2 or the heat-responsive coating 36B-1, 36B-2 described herein. When the heat-responsive coating 36B-1, 36B-2 is used, the coating 36B-1, 36B-2 may be selected from the group consisting of polylactic acid, poly(lactic-co-glycolic) acid, polycaprolactone, agarose, wax, poly(acrylamide-co-acrylonitrile), poly(N-isopropylacrylamide), cyclodextrins, polyethylene glycol homopolymer, polyethylene glycol graft copolymer, polyethylene block copolymer, and a combination thereof. The wax, when used, may be any suitable example of a wax provided herein.

The substrate 16, 26 may be affixed directly to, and thus be in physical contact with, the CMOS chip 94 through one or more securing mechanisms (e.g., adhesive, bond, fasteners, and the like). It is to be understood that the substrate 16, 26 may be removably coupled to the CMOS chip 94.

The CMOS chip 94 includes a plurality of stacked layers 96 including, for example, silicon layer(s), dielectric layer(s), metal-dielectric layer(s), metal layer(s), etc.). The stacked layers 96 make up the device circuitry, which includes detection circuitry.

The CMOS chip 94 includes optical components, such as optical sensor(s) 98 and optical waveguide(s) 100. The optical components are arranged such that each optical sensor 98 at least substantially aligns with, and thus is operatively associated with, a single optical waveguide 100 and a single reactive region 29A or 29B of the flow cell 10′. However, in other examples, a single optical sensor 98 may receive photons through more than one optical waveguide 100 and/or from more than one reactive region 29A, 29B. In these other examples, the single optical sensor 98 is operatively associated with more than one optical waveguide 100 and/or more than one reactive region 29A, 29B.

As used herein, a single optical sensor 98 may be a light sensor that includes one pixel or more than one pixel. As an example, each optical sensor 98 may have a detection area that is less than about 50 μm². As another example, the detection area may be less than about 10 μm². As still another example, the detection area may be less than about 2 μm². In the latter example, the optical sensor 98 may constitute a single pixel. An average read noise of each pixel of the optical sensor 98 may be, for example, less than about 150 electrons. In other examples, the read noise may be less than about 5 electrons. The resolution of the optical sensor(s) 98 may be greater than about 0.5 megapixels (Mpixels). In other examples, the resolution may be greater than about 5 Mpixels, or greater than about 10 Mpixels.

Also as used herein, a single optical waveguide 100 may be a light guide including a cured filter material that i) filters the excitation light 104 (propagating from an exterior of the flow cell 10′ into the flow channel 12), and ii) permits the light emissions (not shown, resulting from reactions at the reactive region(s) 29A, 29B) to propagate therethrough toward corresponding optical sensor(s) 98. In an example, the optical waveguide 100 may be, for example, an organic absorption filter. As a specific example, the organic absorption filter may filter excitation light 104 of about 532 nm wavelength and permit light emissions of about 570 nm or more wavelengths. The optical waveguide 100 may be formed by first forming a guide cavity in a dielectric layer 106, and then filling the guide cavity with a suitable filter material.

The optical waveguide 100 may be configured relative to the dielectric material 106 in order to form a light-guiding structure. For example, the optical waveguide 100 may have a refractive index of about 2.0 so that the light emissions are substantially reflected at an interface between the optical waveguide 100 and the surrounding dielectric material 106. In certain examples, the optical waveguide 100 is selected such that the optical density (OD) or absorbance of the excitation light 104 is at least about 4 OD. More specifically, the filter material may be selected and the optical waveguide 100 may be dimensioned to achieve at least 4 OD. In other examples, the optical waveguide 100 may be configured to achieve at least about 5 OD or at least about 6 OD.

In this example, the substrate 16, 26 functions as a passivation layer. At least a portion of the passivating substrate 16, 26 is in contact with a first embedded metal layer 112 of the CMOS chip 94 and also with an input region 110 of the optical waveguide 100. The contact between the passivating substrate 16, 26 and the first embedded metal layer 112 may be direct contact or may be indirect contact through a shield layer 114.

The substrate 16, 26 (passivation layer) may provide one level of corrosion protection for the embedded metal layer 112 of the CMOS chip 94 that is closest in proximity to the substrate 16, 26. In this example, the substrate 16, 26 may include a passivation material that is transparent to the light emissions resulting from reactions at the reactive region 29A, 29B (e.g., visible light), and that is at least initially resistant to the fluidic environment and moisture that may be introduced into or present in the flow channel 12. An at least initially resistant material acts as an etch barrier to high pH reagents (e.g., pH ranging from 8 to 14) and as a moisture barrier. Examples of suitable materials for the substrate 16, 26 of the flow cell 10′ include silicon nitride (Si₃N₄), silicon oxide (SiO₂), tantalum pentoxide (TaO₅), hafnium oxide (HfO₂), boron doped p+ silicon, or the like. The thickness of the substrate 16, 26 may vary depending, in part upon the sensor dimensions. In an example, the thickness of the substrate 16, 26 ranges from about 100 nm to about 500 nm.

The flow cell 10′ also includes a lid 116 that is operatively connected to the substrate 16, 26 to partially define the flow channel 12 between the substrate 16, 26 (and the reactive region(s) 29A, 29B therein or thereon) and the lid 116. The lid 116 may be any material that is transparent to the excitation light 104 that is directed toward the reactive region(s) 29A, 29B. As examples and as described in regard to the flow cell 10 of FIG. 1A through FIG. 1C, the lid 116 may include glass (e.g., borosilicate, fused silica, etc.), plastic, etc. A commercially available example of a suitable borosilicate glass is D 263®, available from Schott North America, Inc. Commercially available examples of suitable plastic materials, namely cyclo olefin polymers, are the ZEONOR® products available from Zeon Chemicals L.P.

In an example, the lid 116 of the flow cell 10′ may be a substantially rectangular block having an at least substantially planar exterior surface 118 and an at least substantially planar interior surface 120 that defines a portion of the flow channel 12. The block may be mounted onto the material 64. Alternatively, the block may be etched so that the lid 116 defines the both the top and sidewalls of the channel 12. In these instances, a thin layer of the material 64 may bind the lid 116 to the substrate 16, 18. In an example, a recess may be etched into the transparent block. When the etched block is mounted to the substrate 16, 26, the recess may become the flow channel 12.

The lid 116 may include inlet and outlet ports 122, 124 that are configured for fluidic engagement with other ports (not shown) to direct fluid(s) into the flow channel 12 (e.g., from a reagent cartridge or other fluid storage system component) and out of the flow channel 12 (e.g., to a waste removal system).

As noted, the lid 116 may be physically connected to the substrate 16, 26 through material 64. The material 64 is/are coupled to a portion the surface of the substrate 16, 26, and extends between those portions of the lid 116 that are to contact the substrate 16, 18. In some examples, the material 64 includes a curable adhesive layer that bonds the lid 116 to the substrate 16, 26 (at a portion of its surface).

The flow cell 10′ also includes the heating mechanism(s) 74 or electrode(s) 76 (again, which are not shown in FIG. 4). The selection of the heating mechanism(s) 74 or electrode(s) 76 and its positioning within the flow cell 10′ will depend upon the removable coating that is used.

When the removable coating is the heat-responsive coating 36B-1, 36B-2, the heating mechanism(s) 74 is/are used, and the heating mechanism(s) 74 may be incorporated into the lid 116, the depressions 22, the substrate 16, 18, or within the CMOS chip 94. In one example, each of the plurality of reactive regions 29A, 29B may align with one or more heating mechanisms 74 included in the CMOS chip 94 itself (e.g., sandwiched between the optical waveguide 100 and the substrate 16 or 26).

The one or more heating mechanisms 74 may include one or more electrode materials that are capable of converting electricity to a suitable amount of thermal energy to heat up the heat-responsive coating 36B-1, 36B-2. When used, the one or more electrode material(s) has/have a thickness ranging from about 1 nm to about 20 nm, and thus the one or more electrode material(s) is/are transparent to the wavelengths that may be used during a sequencing operation. The one or more electrode material(s) suitable for the heating mechanism 74 may include a suitable conductive material or alloy, such as copper, graphite, titanium, silver, platinum, tungsten, or the like.

When the removable coating is the gas-dissolvable coating 36A-1, 36A-2, the electrode(s) 76 is/are used, and the electrode(s) 76 may be incorporated into the lid 116 or in the depressions 22. In one example, the electrode(s) 76 is/are carbon anodes.

It is to be understood that because the emissions from the reaction taking place at the reactive regions 29A, 29B in the flow cell 10′ are directed toward the optical waveguide 100, any heating mechanism 74 or electrode 76 that is in the depression 22 or the substrate 16, 18 should be i) optically transparent if it is positioned between the optical waveguide 100 and the substrate 16, 18 or on the bottom of the depressions 22 or ii) should be incorporated along the sidewalls of the depression 22.

When used, each of the heating mechanisms 74 or electrodes 76 spatially align(s) with at least one reactive region 29A, 29B. In one example, each reactive region 29A, 29B aligns with one heating mechanism 74 or electrode 76, and thus each reactive region 29A, 29B is independently addressable. As such, a heating mechanism 74 or electrode 76 aligned with one reactive region 29A or 29B may be activated, while a heating mechanism 74 or electrode 76 aligned with the other reactive region 29B or 29A remains inactive. In another example, a sub-set of reactive regions 29A, 29B (relative to all of the reactive regions in the flow cell 10′) align with one heating mechanism 74 or electrode 76, and thus the reactive regions 29A, 29B in the sub-set are addressable together. For one example, two heating mechanisms 74 may each individually address several, or hundreds, or thousands, or millions of reactive regions 29A, 29B, depending on the number of reactive regions 29A, 29B that are utilized, the space between individual reactive regions 29A, 29B, and the size/number/layout of heating mechanisms 74 or electrodes 76.

In an example of the flow cell 10′ including multiple heating mechanisms 74 that heat multiple reactive regions 29A, 29B, it may be desirable to include a gap that separates each heating mechanism 74 from each other heating mechanism 74. The gap may help to prevent thermal runaway, and thus undesirable heating of a non-activated region 29A or 29B. In an example, the gap ranges from about 1 mm to about 5 mm. The amount of space between adjacent heating mechanisms 74 may also be characterized in terms of a ratio relating the amount of surface area occupied by heating mechanisms 74 to the amount of surface area occupied by gaps between the heating mechanisms 74, based on a total surface area of the lid 116, or the substrate 16, 18, or the CMOS chip 94 where the heating mechanisms 74 are located. In an example, the ratio of heating mechanisms 74 to gaps ranges from about 1:3 to about 3:1. In another specific example, the ratio of heating mechanisms 74 to gaps is about 2:1.

The flow channel 12 of the flow cell 10′ may be sized and shaped to direct a fluid along the reactive region(s) 29A, 29B. The height of the flow channel 12 and other dimensions of the flow channel 12 may be configured to maintain a substantially even flow of the fluid along the reactive region(s) 29A, 29B. The dimensions of the flow channel 12 may also be configured to control bubble formation. In an example, the height (or depth) of the flow channel 12 may range from about 50 μm to about 400 μm. In another example, the height of the flow channel 12 may range from about 80 μm to about 200 μm. It is to be understood that the height of the flow channel 12 may vary, and may be the greatest when the reactive region 29A, 29B is located in a reaction chamber (e.g., depression 22) that is defined in the surface of the substrate 16, 26. In these examples, the depression 22 increases the height of the flow channel 12 at this particular area.

In an example, the reactive region 29A or 29B is at least substantially aligned with the input region 110 of a single optical waveguide 100. As such, light emissions at the reactive region 29A, 29B may be directed into the input region 110, through the waveguide 100, and to an associated optical sensor 98. In other examples, one reactive region 29A, 29B may be aligned with several input regions 110 of several optical waveguides 100. In still other examples, several reactive regions 29A, 29B may be aligned with one input region 110 of one optical waveguide 100.

The embedded metal layer 112 may be any suitable CMOS metal, such as aluminum (Al), aluminum chloride (AlCl), tungsten (W), nickel (Ni), or copper (Cu). The embedded metal layer 112 is a functioning part of the CMOS chip 94, and through the stacked layers 96, is also electrically connected to the optical sensor 98. Thus, the embedded metal layer 112 participates in the detection/sensing operation. The embedded metal layer 112 can also be configured to function as the heating mechanism 74.

It is to be understood that the other optical sensors 98 and associated components may be configured in an identical or similar manner. It is also to be understood, however, that the CMOS chip 94 may not be manufactured identically or uniformly throughout. Instead, one or more optical sensor 98 and/or associated components may be manufactured differently or have different relationships with respect to one another.

The stacked layer 96 may include interconnected conductive elements (e.g., conductors, traces, vias, interconnects, etc.) that can conduct electrical current. The circuitry may be configured for selectively transmitting data signals that are based on detected photons. The circuitry may also be configured for signal amplification, digitization, storage, and/or processing. The circuitry may collect and analyze the detected light emissions and generate data signals for communicating detection data to a bioassay system. The circuitry may also perform additional analog and/or digital signal processing in the CMOS chip 94.

The CMOS chip 94 may be manufactured using integrated circuit manufacturing processes. The CMOS chip 94 may include multiple layers, such as a sensor base/layer (e.g., a silicon layer or wafer). The sensor base may include the optical sensor 98. When the CMOS chip 94 is fully formed, the optical sensor 98 may be electrically coupled to the rest of the circuitry in the stack layer 96 through gate(s), transistor(s), etc. As described, the CMOS chip 94 may further include heating mechanism materials that align with reactive regions 29A, 29B of the flow cell 10′.

The CMOS chip 94 may have a thickness ranging from about 10 μm to about 200 μm. In a specific example, the CMOS chip 94 has a thickness of about 100 μm.

As used in reference to FIG. 4, the term “layer” is not limited to a single continuous body of material unless otherwise noted. For example, the sensor base/layer may include multiple sub-layers that are different materials and/or may include coatings, adhesives, and the like. Furthermore, one or more of the layers (or sub-layers) may be modified (e.g., etched, deposited with material, etc.) to provide the features described herein.

The stacked layer 96 also includes a plurality of metal-dielectric layers. Each of these layers includes metallic elements (e.g., M1-M5, which may be, for example, W (tungsten), Cu (copper), Al (aluminum), or any other suitable CMOS conductive material) and dielectric material 106 (e.g., SiO₂). Various metallic elements M1-M5 and dielectric materials 106 may be used, such as those suitable for integrated circuit manufacturing.

In the example shown in FIG. 4, each of the plurality of metal-dielectric layers L1-L6 includes both metallic elements M1, M2, M3, M4, M5 and dielectric material 106. In each of the layers L1-L6, the metallic elements M1, M2, M3, M4, M5 are interconnected and are embedded within dielectric material 106. In some of the metal-dielectric layers L1-L6, additional metallic elements may also be included. Some of these additional metallic elements may be used to address individual pixels through a row and column selector. The voltages at these elements may vary and switch between about −1.4 V and about 4.4 V depending upon which pixel the device is reading out.

The configuration of the metallic elements M1, M2, M3, M4, M5 and dielectric layer 106 in FIG. 4 is illustrative of the circuitry, and it is to be understood that other examples may include fewer or additional layers and/or may have different configurations of the metallic elements M1-M5.

In the example shown in FIG. 4, the shield layer 114 is in contact with at least a portion of the substrate 16 or the base support 26 of the substrate 18. The shield layer 114 has an aperture at least partially adjacent to the input region 110 of the optical waveguide 100. This aperture enables the reactive region 29A, 29B (and at least some of the light emissions therefrom) to be optically connected to the waveguide 100. It is to be understood that the shield layer 114 may have an aperture at least partially adjacent to the input region 110 of each optical waveguide 100. The shield layer 114 may extend continuously between adjacent apertures.

The shield layer 114 may include any material that can block, reflect, and/or significantly attenuate the light signals that are propagating through the flow channel 12. The light signals may be the excitation light 104 and/or the light emissions from the reactive region(s) 29A, 29B. As an example, the shield layer 114 may be tungsten (W).

It is to be understood that the flow cell 10′ may also be used for optical detection.

Any of the example coatings 36 set forth herein may be used in combination in order to obtain multi-layer coatings. Examples of some multi-layer removable coatings 38A, 38B are shown in FIG. 6A. In this example, at least one of the plurality of independently removable coatings includes a plurality of sub-layers (i.e., is a multi-layer removable coating 38A, 38B); and the plurality of sub-layers defines the removal characteristic of the at least one of the plurality of independently removable coatings.

In this example, the multi-layer removable coatings 38A, 38B require sequential heat or gas treatments in order to reveal an underlying reactive region 29A, 29B. The sequential treatments will depend upon the sub-layers, e.g., coatings 361, 362, 363, that are included in the stack. The sub-layers in each of the multi-layer removable coatings 38A, 38B may be selected so that some of the reactive regions 29A, 29B remain coated with at least some sub-layers of the multi-layer removable coating 38A or 38B, even when other sub-layers of the multi-layer removable coating 38A or 38B are removed or when another multi-layer removable coating 38B or 38A is completely removed. In the example show in FIG. 6A, the removal of the multi-layer coating 38A involves the sequential heating or gas exposure of coating 362 and then coating 361, while the removal of multi-layer coating 38B involves the sequential heating or gas exposure of coatings 363, 362, and then 361.

Any of the example coatings set forth herein may have variable thicknesses in order to alter the removal characteristic of the independently removable coatings 36. An example is shown in FIG. 6B. In this example, each of the coatings 364, 365, 366, is made up of the same heat-responsive or gas dissolvable coating, but has a different thickness T₁, T₂, T₃. As such, while the coatings 364, 365, 366 are susceptible to the same removal characteristic (heat or gas), the rates at which the coatings 364, 365, 366 dissolve or melt are different due to the different thicknesses T₁, T₂, T₃. In one example, the thickness T₁<the thickness T₂<the thickness T₃, and T₁ ranges from about 10 nm to about 500 nm, T₂ ranges from about 100 nm to about 500 nm, and T₃ ranges from about 500 nm to about 1000 nm.

While the example coatings 361, 362, 363, 364, 365, 366 are shown over the protrusions 24, it is to be understood that these coatings 361, 362, 363, 364, 365, 366 may be used in the depressions 22 as well.

### Methods for Using Flow Cells

Two examples of a method for using flow cells 10, 10′ that include the gas-dissolvable coatings 36A-1, 36A-2 are depicted in FIG. 5 at A., B., C., D., and E. and at A., B., F., G., and E. These example methods generally include i) selectively removing at least one of a plurality of independently removable coatings 36 respectively positioned over each of a plurality of reactive regions 29A, 29B spatially separated from one another across a substrate 16, 18, thereby exposing at least one of the plurality of reactive regions 29A, 29B and a reactive entity 34 at the at least one of the plurality of reactive regions 29A, 29B; wherein each of the plurality of reactive regions 29A, 29B includes a polymeric hydrogel layer 32 and a reactive entity 34 attached to the polymeric hydrogel layer 32; and wherein at least one of the plurality of independently removable coatings 36 is the gas-dissolvable coating 36A-1, 36A-2; and ii) initiating a reaction involving the reactive entity 34.

In FIG. 5 at A., a first gas-dissolvable coating 36A-1 is shown as being applied over the reactive region 29A in one of the depressions 22, and a second gas-dissolvable coating 36A-2 is shown as being applied over the reactive region 29B in another of the depressions 22. The gas-dissolvable coating 36A-1 positioned over the reactive region 29A may be same as the gas-dissolvable coating 36A-2 positioned over the reactive region 29B, or these two gas-dissolvable coatings 36A-1, 36A-2 may be different (e.g., may be susceptible to removal under differing gas conditions).

The gas dissolvable coating(s) 36A-1, 36A-2 that is/are used in these example methods may include any of the suitable materials set forth herein.

At A., a reactive gas is used to selectively remove the gas-dissolvable coating 36A-1 that overlies the reactive entity 34A within reactive region 29A. The reactive gas that is used to remove this gas-dissolvable coating 36A-1 will depend upon the material used for the coating 36A-1. The reactive gas used to remove the coating 36A-1 overlying the reactive entity 34A may be carbon dioxide gas, or oxygen gas, or a reactive oxygen species.

In some examples, the reactive gas that is used to remove the coating 36A-1 at A. is generated using an electrode 76, and the electrode 76 may align with the reactive region 29A, as shown. The electrode 76 may be any electrode material that is included in the lid 116 that forms a part of the flow cell 10 (when used), any electrode material that is included/embedded in the substrate 16 or the layer 28 of the substrate 18 of the flow cell 10, or any electrode material that is included in a CMOS chip 94 that is coupled to the flow cell (thereby forming a flow cell 10′), or any electrode material that has been deposited within the depression(s) 22 that are defined in the flow cell 10, such that the electrode covers a bottom surface of the depression(s) 22.

In some examples, a gas-generative species (not shown) may be embedded within the reactive region 29A. In one of these examples, localized heating (induced through the heating mechanism 74) may be used to degrade the gas-generative species, thereby producing reactive gas when the gas-generative species reaches a predetermined temperature. In this example, the gas-generative species may be baking soda (sodium bicarbonate, NaHCO₃), which degrades to carbon dioxide gas at about 80° C. and can then be used to remove the coating 36A-1 over the reactive region 29A. Alternatively in this example, the gas-generative species may be sodium chlorate or lithium perchlorate, either of which can be degraded to generate oxygen gas at temperatures of about 300° C. or higher. Further in this example, the heating mechanism 74 may also function as an electrode 76.

In some other examples in which the gas-generative species is embedded within the reactive region 29A, the gas-generative species may be converted to gas by lowering the pH of the gas-generative species (e.g., through exposure to a suitable acid). In these examples, the acid (or another source of pH-lowering H+ ions) is generated in the vicinity of the anode, and the acid or source of pH-lowering H+ ions deprotects the gas-generative species to liberate gaseous byproducts (that can be used to remove the coating 36A-1). In a specific example, the gas-generative species includes a carbamate moiety (e.g., a tert-butyl carbamate), and exposure of the gas-generative species to an acid (such as HCl, trifluoroacetic acid (TFA)) deprotects the carbamate and generates gas (CO₂). In some instances, the carbamate moiety is included in the gas-dissolvable coating 36A-1, and deprotection of the tert-butyl carbamate groups renders the coating 36A-1 more hydrophilic (and thus dissolvable).

As another example, the reactive gas that is used to remove the coating 36A-1 overlying the reactive entity 34A is generated using a carbon anode. In this example, the carbon anode may be included within the depression 22 (containing reactive entity 34A), or in a lid 116 that forms a part of the flow cell 10, 10′.

In yet another example, gases that are initially trapped within the depression 22 (e.g., within the depression 22 that includes reactive entity 34A) may be locally burst using locally applied acoustics or using heat generated by the heating mechanism 74.

In still another example, a controlled stream of reactive gas from an external source may be directed towards the gas-dissolvable coating 36A-1 covering the reactive region 29A.

As such, the gas that is used to remove the coating 36A-1 from the reactive region 29A may be generated by (the degradation of) a gas-generative species within the reactive regions 29A, or from an electrode 76 (such as a carbon anode) that aligns with the reactive region 29A, or using a directed stream of gas from an external source.

In some instances, and as described in reference to FIG. 2A and FIG. 2B, selectively removing the independently removable coating 36A-1 (e.g., from within the reactive region 29A) involves exposing the independently removable coating 36A-1 to a reactive gas and then water in the presence of an inert gas, thereby dissolving the at least one of the plurality of independently removable coatings 36A-1. The removal process that is used to remove the coating 36A-1 from over the reactive region 29A may leave the other coating 36A-2 over the reactive region 29B substantially intact.

At B., a template strand 40 of a first library of template strands is introduced and seeds to the reactive entity 34A that has been exposed by the removal of the coating 36A-1 from the reactive region 29A.

In some examples, the reactive entity 34 in each of the plurality of reactive regions 29A, 29B is a primer set. As described, the primer set may be the same in each of the plurality of reactive regions 29A, 29B, or the primer set of at least one of the plurality of reactive regions 29A, 29B may be different than the primer set of at least one other of the plurality of reactive regions 29B, 29A. In the examples depicted in FIG. 5, the reactive entity 34A within the reactive region 29A is shown as being different than the reactive entity 34B within the reactive region 29B. As examples, the reactive entities 34A, 34B in the two adjacent depressions 22 may be two different primer sets. In other examples, however, the same reactive entity 34A or 34B may be respectively applied in two adjacent depressions 22.

When the method continues at C., the seeded template strand 40 will occupy a certain density of the reactive entity 34A. Once seeding takes place, additional reactive gas is used to selectively remove the gas-dissolvable coating 36A-2 that overlies the reactive entity 34B. The gas-dissolvable coating 36A-2 overlying the reactive entity 34B may be removed in any manner set forth herein in regard to the coating 36A-1 overlying the reactive entity 34A.

At D., another template strand 40′ of a second library of template strands is introduced and seeds to the reactive entity 34B (that has been exposed by the removal of the gas-dissolvable coating 36A-2 from the reactive region 29B). In an example, the template strand 40′ does not include an adapter for hybridizing to the reactive entity 34A, and thus will not seed in the depression(s) 22 containing the reactive entity 34A. In this example, different depressions 22 may individually contain different reactive entities 34A, 34B (e.g., different primer sets). In another example, the depressions 22 that are exposed during the seeding of the template strands 40 may be smaller than the depressions 22 that are exposed during the seeding of the template strands 40′. In these instances, the template strands 40′ may be sterically blocked from seeding in the depressions 22 containing the template strands 40.

Each of the seeded template strands 40, 40′ is then respectively amplified and clustered across the respective reactive entities 34A, 34B, as shown in E. This generates first library template amplicons 42 in at least one of the depressions 22 (e.g., at reactive region 29A) and second library template amplicons 42′ in at least one other of the depressions 22 (e.g., at reactive region 29B).

When the method continues at F. from B., the seeded template strand 40 is amplified and clustered across the respective reactive entities 34A to generate the first library template amplicons 42. Once the cluster of amplicons 42 is generated using the reactive entity 34A, additional reactive gas is used to selectively remove the gas-dissolvable coating 36A-2 that overlies the reactive entity 34B. Any example of removal processes set forth in regard to the coating 36A-2 overlying the reactive entity 34B (at C.) may also be used to remove the coating 36A-2 overlying the reactive entity 34B (at F.). At G., another template strand 40′ of a second library of template strands is introduced and seeds to the reactive entity 34B. The seeded template strand 40′ is then amplified and clustered across the reactive entity 34B, as shown in E. This generates the second library template amplicons 42′ in the depression 22 including the reactive entity 34B.

Two examples of a method for using flow cells 10, 10′ that include the heat-responsive coatings 36B-1, 36B-2 are also depicted in FIG. 5 at A. through E. and at A., B., F., G., and E. These example methods generally include i) selectively removing at least one of a plurality of independently removable coatings 36 respectively positioned over each of a plurality of reactive regions 29A, 29B spatially separated from one another across a substrate 16, 18 by activating one or more heating mechanisms 74, thereby exposing at least one of the plurality of reactive regions 29A, 29B and a reactive entity 34 at the at least one of the plurality of reactive regions 29A, 29B; wherein each of the plurality of reactive regions 29A, 29B includes a polymeric hydrogel layer 32 and a reactive entity 34 attached to the polymeric hydrogel layer 32; wherein at least one of the plurality of independently removable coatings 36 is the heat-responsive coating 36B-1, 36B-2; and wherein each of the plurality of reactive regions 29A, 29B aligns with the one or more heating mechanisms 74; and ii) initiating a reaction involving the reactive entity 34.

In each of these methods, a first heat-responsive coating 36B-1 is shown as being applied over the reactive region 29A in each of the depressions 22, and a second heat-responsive coating 36B-2 is shown as being applied over the reactive region 29B in each of the depressions 22. The heat-responsive coating 36B-1 positioned over the reactive region 29A may be same as the heat-responsive coating 36B-2 positioned over the reactive region 29B, or these two heat-responsive coatings 36B-1, 36B-2 may be different (e.g., may be susceptible to removal under differing conditions). In the example shown in FIG. 5, the reactive entity 34A in one of the depressions 22 (e.g., at reactive region 29A) is shown as being different than the reactive entity 34B in the other of the depressions 22 (e.g., at reactive region 29B). As one example, the reactive entities 34A, 34B in the two adjacent depressions 22 may be two different primer sets. In other examples, however, the same reactive entity 34A or 34B may be respectively applied in two adjacent depressions 22.

The heat-responsive coating(s) 36B-1, 36B-2 that is/are used in these example methods may include any of the suitable materials set forth herein.

At A., a heating mechanism 74 is used to generate heat that selectively removes the heat-responsive coating 36B-1 that overlies the reactive entity 34A within reactive region 29A. As described, the heating mechanism 74 may be any heating mechanism material that is included in the lid 116 that forms a part of the flow cell 10 (when used), any heating mechanism material that is included/embedded in the substrate 16 or the layer 28 of the substrate 18 of the flow cell 10, any heating mechanism material that is included in a CMOS chip 94 that is coupled to the flow cell (thereby forming a flow cell 10′), or any heating mechanism material that has been deposited within the depression(s) 22 that are defined in the flow cell 10, such that the heating mechanism material covers a bottom surface of the depression(s) 22 or is embedded in sidewalls of the depressions 22.

The amount of heat that is used to remove the coating 36B-1 overlying the reactive entity 34A via the heating mechanism 74 may depend, in part, upon the material included in the heat-responsive coating 36B-1 overlying the reactive region 29A and the type/placement of the heating mechanism 74. Accordingly, in some instances, the heat that is generated by the heating mechanism 74 may be tuned (in terms of magnitude) to facilitate the removal of the heat-responsive coating 36B-1 overlying the reactive region 29A.

In some examples, selectively removing the at least one of the plurality of independently removable coatings 36 (e.g., 36B-1) involves exposing the at least one of the plurality of independently removable coatings 36 to heat generated by the one or more heating mechanisms 74, thereby rendering the at least one of the plurality of independently removable coatings 36 soluble in an aqueous solvent.

At B., a template strand 40 of a first library of template strands is introduced and seeds to the reactive entity 34A that has been exposed by the removal of the coating 36B-1 from the reactive region 29A.

When the method continues at C., the seeded template strand 40 will occupy a certain density of the reactive entity 34A. Once seeding takes place, additional heat (e.g., generated by the heating mechanism 74) is used to selectively remove the heat-responsive coating 36B-2 that overlies the reactive entity 34B. The heat that is generated by the heating mechanism 74 may be tuned (in terms of magnitude) to facilitate the removal of the heat-responsive coating 36B-2 overlying the reactive region 29B.

The heat-responsive coating 36B-2 overlying the reactive entity 34B may be removed by either the same or a different heating mechanism 74 than the heating mechanism 74 used to remove the heat-responsive coating 36B-1. In a specific example, the heat-responsive coating 36B-2 overlying the reactive entity 34B is removed using a separate heating mechanism 74 than the heating mechanism 74 that is used to remove the heat-responsive coating 36B-1 overlying the reactive entity 34A. In this example, the two heating mechanisms 74 may be spaced apart from one another by a gap ranging from about 0.5 mm to about 5 mm.

At D., another template strand 40′ of a second library of template strands is introduced and seeds to the reactive entity 34B (that has been exposed by the removal of the heat-responsive coating 36B-2 from the reactive region 29B). In an example, the template strand 40′ does not include an adapter for hybridizing to the reactive entity 34A, and thus will not seed in the depression(s) 22 containing the reactive entity 34A. In this example, different depressions 22 may individually contain different reactive entities 34A, 34B (e.g., different primer sets). In another example, the depressions 22 that are exposed during the seeding of the template strands 40 may be smaller than the depressions 22 that are exposed during the seeding of the template strands 40′. In these instances, the template strands 40′ may be sterically blocked from seeding in the depressions 22 containing the template strands 40.

Each of the seeded template strands 40, 40′ is then respectively amplified and clustered across the respective reactive entities 34A, 34B, as shown in E. This generates first library template amplicons 42 in at least one of the depressions 22 (e.g., at reactive region 29A) and second library template amplicons 42′ in at least one other of the depressions 22 (e.g., at reactive region 29B).

When the method continues at F. (from B.), the seeded template strand 40 is amplified and clustered across the respective reactive entities 34A to generate the first library template amplicons 42. Once the cluster of amplicons 42 is generated using the reactive entity 34A, additional heat is used to selectively remove the heat-responsive coating 36B-2 that overlies the reactive entity 34B. At G., another template strand 40′ of a second library of template strands is introduced and seeds to the reactive entity 34B. The seeded template strand 40′ is then amplified and clustered across the reactive entity 34B, as shown in E. This generates the second library template amplicons 42′ in at least one other of the depressions 22.

While FIG. 5 depicts specific examples with regard to the type of reaction that is performed, it is to be understood that different reactions can be performed when different reactive entities 34 are utilized. As one example, tagmentation may be performed when the reactive entities 34 are transposome complexes.

### Additional Notes

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

Reference throughout the specification to “one example”, “another example”, “an example”, and so forth, means that a particular element (e.g., feature, structure, and/or characteristic) described in connection with the example is included in at least one example described herein, and may or may not be present in other examples. In addition, it is to be understood that the described elements for any example may be combined in any suitable manner in the various examples unless the context clearly dictates otherwise.

While several examples have been described in detail, it is to be understood that the disclosed examples may be modified. Therefore, the foregoing description is to be considered non-limiting.

What is claimed is:

1. A flow cell, comprising:
a substrate;
a plurality of reactive regions spatially separated from one another across the substrate, each of the plurality of reactive regions including:
a polymeric hydrogel layer; and
a reactive entity attached to the polymeric hydrogel layer; and
a plurality of independently removable coatings respectively positioned over each of the plurality of reactive regions;
wherein at least one of the plurality of independently removable coatings is a gas-dissolvable coating.

2. The flow cell as defined in claim 1, wherein the reactive entity in each of the plurality of reactive regions is a primer set.

3. The flow cell as defined in claim 2, wherein the primer set is the same in each of the plurality of reactive regions.

4. The flow cell as defined in claim 2, wherein the primer set of at least one of the plurality of reactive regions is different than the primer set of at least one other of the plurality of reactive regions.

5. The flow cell as defined in claim 1, wherein:
the substrate includes a plurality of depressions;
each of the plurality of reactive regions is positioned within a respective one of the plurality of depressions; and
each of the plurality of independently removable coatings covers a respective one of the plurality of reactive regions.

6. The flow cell as defined in claim 1, wherein:
the substrate includes a plurality of protrusions;
each of the plurality of reactive regions is positioned at a respective one of the plurality of protrusions; and
each of the plurality of independently removable coatings covers a respective one of the plurality of protrusions.

7. The flow cell as defined in claim 1, wherein the gas-dissolvable coating is selected from the group consisting of an amine-based coating, an amidine-based coating, a guanidine-based coating, an oxygen-responsive copolymer, an oxidation-responsive copolymer, and combinations thereof.

8. A method, comprising:
selectively removing at least one of a plurality of independently removable coatings respectively positioned over each of a plurality of reactive regions spatially separated from one another across a substrate, thereby exposing at least one of the plurality of reactive regions and a reactive entity at the at least one of the plurality of reactive regions;
wherein each of the plurality of reactive regions includes a polymeric hydrogel layer and a reactive entity attached to the polymeric hydrogel layer; and
wherein at least one of the plurality of independently removable coatings is a gas-dissolvable coating; and
initiating a reaction involving the reactive entity.

9. The method as defined in claim 8, wherein selectively removing the at least one of the plurality of independently removable coatings involves exposing the at least one of the plurality of independently removable coatings to a reactive gas and water in the presence of an inert gas, thereby dissolving the at least one of the plurality of independently removable coatings.

10. The method as defined in claim 8, wherein:
the substrate includes a plurality of depressions;
each of the plurality of reactive regions is positioned within a respective one of the plurality of depressions; and
each of the plurality of independently removable coatings covers a respective one of the plurality of reactive regions.

11. The method as defined in claim 8, wherein:
the substrate includes a plurality of protrusions;
each of the plurality of reactive regions is positioned at a respective one of the plurality of protrusions; and
each of the plurality of independently removable coatings covers a respective one of the plurality of protrusions.

12. The method as defined in claim 8, wherein the reactive entity in each of the plurality of reactive regions is a primer set.

13. The method as defined in claim 12, wherein the primer set is the same in each of the plurality of reactive regions.

14. The method as defined in claim 12, wherein the primer set of at least one of the plurality of reactive regions is different than the primer set of at least one other of the plurality of reactive regions.

15. The method as defined in claim 8, wherein the gas-dissolvable coating is selected from the group consisting of an amine-based coating, an amidine-based coating, a guanidine-based coating, an oxygen-responsive copolymer, an oxidation-responsive copolymer, and combinations thereof.

16. A flow cell, comprising:
a substrate;
a plurality of reactive regions spatially separated from one another across the substrate, each of the plurality of reactive regions including:
a polymeric hydrogel layer; and
a reactive entity attached to the polymeric hydrogel layer;
a heating mechanism aligned with at least one of the plurality of reactive regions; and
a plurality of independently removable coatings respectively positioned over each of the plurality of reactive regions;
wherein at least one of the plurality of independently removable coatings is a heat-responsive coating.

17. The flow cell as defined in claim 16, wherein the reactive entity in each of the plurality of reactive regions is a primer set.

18. The flow cell as defined in claim 17, wherein the primer set is the same in each of the plurality of reactive regions.

19. The flow cell as defined in claim 17, wherein the primer set of at least one of the plurality of reactive regions is different than the primer set of at least one other of the plurality of reactive regions.

20. The flow cell as defined in claim 16, wherein:
the substrate includes a plurality of depressions;
each of the plurality of reactive regions is positioned within a respective one of the plurality of depressions;
each of the plurality of independently removable coatings covers a respective one of the plurality of reactive regions.

21. The flow cell as defined in claim 16, wherein:
the substrate includes a plurality of protrusions;
each of the plurality of reactive regions is positioned at a respective one of the plurality of protrusions; and
each of the plurality of independently removable coatings covers a respective one of the plurality of protrusions.

22. The flow cell as defined in claim 16, wherein the heat-responsive coating is selected from the group consisting of polylactic acid, poly(lactic-co-glycolic) acid, polycaprolactone, agarose, wax, poly(acrylamide-co-acrylonitrile), poly(N-isopropylacrylamide), cyclodextrins, polyethylene glycol homopolymer, polyethylene glycol graft copolymer, polyethylene block copolymer, and a combination thereof.

23. A method, comprising:
selectively removing at least one of a plurality of independently removable coatings respectively positioned over each of a plurality of reactive regions spatially separated from one another across a substrate by activating at least one heating mechanism, thereby exposing at least one of the plurality of reactive regions and a reactive entity at the at least one of the plurality of reactive regions;
wherein each of the plurality of reactive regions includes a polymeric hydrogel layer and a reactive entity attached to the polymeric hydrogel layer;
wherein at least one of the plurality of independently removable coatings is a heat-responsive coating; and
wherein at least one of the plurality of reactive regions aligns with the at least one heating mechanism; and
initiating a reaction involving the reactive entity.

24. The method as defined in claim 23, wherein selectively removing the at least one of the plurality of independently removable coatings involves exposing the at least one of the plurality of independently removable coatings to heat generated by the at least one heating mechanism, thereby rendering the at least one of the plurality of independently removable coatings soluble in an aqueous solvent.

25. The method as defined in claim 23, wherein:
the substrate includes a plurality of depressions;
each of the plurality of reactive regions is positioned within a respective one of the plurality of depressions; and
each of the plurality of independently removable coatings covers a respective one of the plurality of reactive regions.

26. The method as defined in claim 23, wherein:
the substrate includes a plurality of protrusions;
each of the plurality of reactive regions is positioned at a respective one of the plurality of protrusions; and
each of the plurality of independently removable coatings covers a respective one of the plurality of protrusions.

27. The method as defined in claim 23, wherein the reactive entity in each of the plurality of reactive regions is a primer set.

28. The method as defined in claim 27, wherein the primer set is the same in each of the plurality of reactive regions.

29. The method as defined in claim 27, wherein the primer set of at least one of the plurality of reactive regions is different than the primer set of at least one other of the plurality of reactive regions.

30. The method as defined in claim 23, wherein the heat-responsive coating is selected from the group consisting of polylactic acid, poly(lactic-co-glycolic) acid, polycaprolactone, agarose, wax, poly(acrylamide-co-acrylonitrile), poly(N-isopropylacrylamide), cyclodextrins, polyethylene glycol homopolymer, polyethylene glycol graft copolymer, polyethylene block copolymer, and a combination thereof.


SEQUENCE LISTING (13 sequences)

SEQ ID NO: 1 - DNA, 29 bp, synthetic construct
  source 1..29: mol_type = other DNA
  modified_base 23: mod_base = OTHER; note = uracil
aatgatacggcgaccaccgagatctacac

SEQ ID NO: 2 - DNA, 29 bp, synthetic construct
  source 1..29: mol_type = other DNA
  modified_base 23: mod_base = i
aatgatacggcgaccaccgaganctacac

SEQ ID NO: 3 - DNA, 29 bp, synthetic construct
  source 1..29: mol_type = other DNA
  modified_base 23: mod_base = OTHER; note = alkene-thymidine
aatgatacggcgaccaccgaganctacac

SEQ ID NO: 4 - DNA, 24 bp, synthetic construct
  source 1..24: mol_type = other DNA
  modified_base 22: mod_base = OTHER; note = 8-oxoguanine
caagcagaagacggcatacganat

SEQ ID NO: 5 - DNA, 24 bp, synthetic construct
  source 1..24: mol_type = other DNA
  modified_base 20: mod_base = OTHER; note = 8-oxoguanine
caagcagaagacggcatacnagat

SEQ ID NO: 6 - DNA, 24 bp, synthetic construct
  source 1..24: mol_type = other DNA
  modified_base 20: mod_base = OTHER; mod_base = OTHER; note = 8-oxoguanine
  modified_base 23: mod_base = OTHER; note = 8-oxoguanine
caagcagaagacggcatacnanat

SEQ ID NO: 7 - DNA, 24 bp, synthetic construct
  source 1..24: mol_type = other DNA
  modified_base 22: mod_base = OTHER; note = uracil
caagcagaagacggcatacgatat

SEQ ID NO: 8 - DNA, 24 bp, synthetic construct
  source 1..24: mol_type = other DNA
  modified_base 20: mod_base = OTHER; note = uracil
caagcagaagacggcatactagat

SEQ ID NO: 9 - DNA, 29 bp, synthetic construct
  source 1..29: mol_type = other DNA
  modified_base 23: mod_base = OTHER; note = allyl-T
aatgatacggcgaccaccgaganctacac

SEQ ID NO: 10 - DNA, 35 bp, synthetic construct
  source 1..35: mol_type = other DNA
gctggcacgtccgaacgcttcgttaatccgttgag

SEQ ID NO: 11 - DNA, 35 bp, synthetic construct
  source 1..35: mol_type = other DNA
cgtcgtctgccatggcgcttcggtggatatgaact

SEQ ID NO: 12 - DNA, 35 bp, synthetic construct
  source 1..35: mol_type = other DNA
acggccgctaatatcaacgcgtcgaatccgcaact

SEQ ID NO: 13 - DNA, 34 bp, synthetic construct
  source 1..34: mol_type = other DNA
gccgcgttacgttagccggactattcgatgcagc